(19) United States
(12) Patent Application Publication
(10) Pub. No.: US 2013/0064762 A1
(43) Pub. Date: Mar. 14, 2013

(54) METHODS OF DETECTING AND TREATING CANCERS USING AUTOANTIBODIES

(71) Applicant: Sanford M. Simon, New York, NY (US)

(72) Inventor: Sanford M. Simon, New York, NY (US)

(73) Assignee: The Rockefeller University, New York, NY (US)

(21) Appl. No.: 13/611,599

(22) Filed: Sep. 12, 2012

Related U.S. Application Data
(60) Provisional application No. 61/534,341, filed on Sep. 13, 2011.

(51) Int. Cl. G01N 33/574; G01N 21/64; A61K 49/16; A61K 49/00; A61K 51/10

(52) U.S. Cl. 424/1.49; 424/9.1; 435/7.1; 424/9.34

(57) ABSTRACT

This invention generally relates to a method of identifying pre-neoplastic or neoplastic tissue of a mammal by utilizing autoantibodies that detect the pre-neoplastic or neoplastic tissue. Also described herein are methods of killing pre-neoplastic or neoplastic tissue by either binding toxins to autoantibodies that detect the pre-neoplastic or neoplastic tissue or introducing toxin-conjugated molecules that can in turn recognize the autoantibodies already bound to the pre-neoplastic or neoplastic tissue.

### RELATED APPLICATIONS

This application claims priority to U.S. provisional application Ser. No. 61/534,341, filed Sep. 13, 2011, which is herein incorporated by reference in its entirety.

### BACKGROUND OF THE INVENTION

Cancer is a heterogeneous collection of diseases. Within a given cancer type, each individual tumor is defined by a distinct set of mutations resulting in different molecular profiles that could elicit an immune response. A neoplastic cell may have many mutations throughout its DNA (Lobe et al., 2003). Further, a particular tumor continues to mutate over time, which causes an evolving and diversifying phenotype. This variability complicates diagnosis and therapy.

It has long been thought that the immune system suppresses developing tumors (Ehrlich 1909). This is consistent with the observation that immunosuppressed transplant recipients have higher rates of non-viral associated tumors than the general population (Birkeland 1995; Penn 1995; Penn 1996; Pam 1995). Transformation leads to tumorigenesis when mutations in the cells allow them to escape the effects of the immune system (Dunn et al., 2002). Although the immune system cannot kill the tumor, it may still recognize the transitioning tissue. Autoantibodies can be generated in response to molecules that are associated with tumors. Various groups have identified a number of tumor-associated autoantibodies in the hopes of utilizing them as biomarkers, prognostic factors, and indicators of tumor recurrence. Autoantibodies to NY-ESO-1 have been identified in the sera of patients with esophageal, lung, liver, breast, thyroid, prostate, and colorectal cancers (Akcakanat et al., 2004; Chapman et al., 2008; Fosså et al., 2004; Korangy et al., 2004; Maio et al., 2003; Nakamura et al., 2006; Stockert et al., 1998; Türeci et al., 2006). The sensitivity and specificity of tumor detection in the sera is increased by testing for the presence of a panel of antibodies, rather than a single antibody (Kobold et al., 2010); however, this is still not sufficient for diagnosis in many tumor types. For example, probing for a single autoantibody in the serum gives a positive result in 10-20% of patients with hepatocellular carcinoma. The detection increases to 66% with a panel of ten autoantibodies (Zang and Tan 2010). If a panel is more useful than a single antibody, the entire collection of autoantibodies might be even more effective in detecting tumors.

There is a need for methodology that improves early detection of neoplasia, as well as detection of metastases, unrecognized foci and transformed cells at surgical margins. Such methodology would optimally be able to continue to track tumors even as they change as a consequence of mutagenesis. Early detection increases the possibility of treating a tumor before the development of metastasis, improving patient survival (Lobe et al, 2003). The present invention utilizes autoantibodies to reliably identify pre-neoplastic and neoplastic tissue with high sensitivity and distinguish these from normal tissue. Genetic instability in neoplasias make them a moving target for detection and therapeutics. Numerous therapeutics that are initially efficacious against neoplasia fail as the tumor mutates and changes. The use of the broad spectra of antibodies generated against a neoplasia ensures that individual, or even bulk changes in the tumor, will not allow the neoplasia to escape detection and treatment. Additionally, the immune system has the neoplasia under continuous surveillance and thus able to detect and respond even to large scale changes. This invention can be used for diagnosis and therapy throughout tumorigenesis.

### SUMMARY OF THE INVENTION

This invention is based, at least in part, on the discovery of a method for identifying pre-neoplastic or neoplastic cells or tissue of a mammal by utilizing autoantibodies that detect the pre-neoplastic or neoplastic cells, tissue, or associated antigen. In related embodiments, the invention involves methods of killing pre-neoplastic or neoplastic cells or tissue by either binding toxins to autoantibodies that detect the pre-neoplastic or neoplastic cells or tissue or introducing toxin-conjugated molecules that can in turn recognize the autoantibodies already bound to the pre-neoplastic or neoplastic tissue.

One aspect the invention provides a method of identifying pre-neoplastic or neoplastic tissue of a mammal comprising detecting autoantibodies complexed to antigen at the tissue, wherein an increase in the amount of autoantibodies complexed to antigen at the tissue, as compared to that at a control tissue, is indicative that the tissue is pre-neoplastic or neoplastic.

In certain embodiments, the tissue is in the body of the mammal. The mammal may be human or an animal. The tissue may be in the body of the mammal or explanted from the mammal. The detection may be done intra-operatively or prior to surgery. If the tissue is an explant, the explant may be fresh, frozen or fixed.

In certain embodiments, detection occurs by detecting an increase in the amount of autoantibodies complexed to antigen at the tissue as compared to that of a control sample.

In certain embodiments, the autoantibodies are labeled with a label. In certain embodiments, the label is an optical reporter, a positron-emission tomography reporter, a magnetic resonance imaging reporter, or a biochemical marker.

In certain embodiments, the autoantibodies are detected using an antibody detection reagent. In certain embodiments, the antibody detection reagent is Anti-IgG, Protein A, Protein G, an anti-IgG, Fab(2) fragment of an anti-IgG, Fab(1) of an anti-IgG and a humanized mouse anti-human IgG, peptides that bind to the Fc region of antibodies, small molecules that recognize IgG for the species of interest, and small molecules that bind the Fc region.

In certain embodiments, the autoantibodies and tissue are autologous. In other embodiments, the autoantibodies and tissue are heterologous but are from one or more individuals of the same species.

In certain embodiments, the pre-neoplastic or neoplastic tissue is in the liver. In certain embodiments, the neoplastic tissue is a fibrolamellar hepatocellular carcinoma. In other embodiments, pre-neoplastic or neoplastic tissue is liver, skin, breast, or prostate tissue and/or cancer.

Another aspect of the invention provides a method of identifying pre-neoplastic or neoplastic tissue of a mammal comprising: (i) providing labeled autologous autoantibodies, (ii) contacting the mammal's tissue with the labeled autologous autoantibodies, and (iii) detecting labeled autologous autoantibodies complexed to an antigen at the tissue, wherein an increase in the amount of labeled autoantibodies complexed to antigen at the tissue is indicative that the tissue is pre-neoplastic or neoplastic.

In certain embodiments, the tissue is in the body of the mammal. The mammal may be human or an animal. The tissue may be in the body of the mammal or explanted from the mammal. The detection may be done intra-operatively or prior to surgery. If the tissue is an explant, the explant may be fresh, frozen or fixed.

In certain embodiments, detection occurs by detecting an increase in the amount of autoantibodies complexed to antigen at the tissue as compared to that of a control sample.

In certain embodiments, the autoantibodies are labeled with a label. In certain embodiments, the label is an optical reporter, a positron-emission tomography reporter, a magnetic resonance imaging reporter, or a biochemical marker. In certain embodiments, the label is a reporter that can be detected optically selected such as a positron emission tomography reporter, CT reporter, X-ray reporter, magnetic resonance imaging reporter, luminescence reporter, RAMAN spectroscopy reporter, surface enhanced Raman spectroscopy (SERS) reporter, second harmonic generation reporter, or biochemical detection reporter.

In certain embodiments, the autoantibodies and tissue are autologous. In other embodiments, the autoantibodies and tissue are heterologous but are from one or more individuals of the same species.

In certain embodiments, the pre-neoplastic or neoplastic tissue is in the liver. In certain embodiments, the neoplastic tissue is a fibrolamellar hepatocellular carcinoma. In other embodiments, pre-neoplastic or neoplastic tissue is liver, skin, breast, or prostate tissue and/or cancer.

Another aspect of the invention provides a method of identifying pre-neoplastic or neoplastic tissue of a mammal comprising: (i) contacting the tissue with the labeled autoantibodies, and (ii) detecting autoantibodies complexed to antigen at the tissue, wherein an increase in the amount of autoantibodies complexed to antigen at the tissue is indicative that the tissue is pre-neoplastic or neoplastic.

In certain embodiments, the tissue is in the body of the mammal. The mammal may be human or an animal. The tissue may be in the body of the mammal or explanted from the mammal. The detection may be done intra-operatively or prior to surgery. If the tissue is an explant, the explant may be fresh, frozen or fixed.

In certain embodiments, detection occurs by detecting an increase in the amount of autoantibodies complexed to antigen at the tissue as compared to that of a control sample.

In certain embodiments, the autoantibodies are labeled with a label. In certain embodiments, the label is an optical reporter, a positron-emission tomography reporter, a magnetic resonance imaging reporter, or a biochemical marker. In certain embodiments, the antibody is labeled with a reporter that can be detected optically such as a positron emission tomography reporter, CT reporter, X-ray reporter, magnetic resonance imaging reporter, luminescence reporter, RAMAN spectroscopy reporter, surface enhanced Raman spectroscopy (SERS) reporter, second harmonic generation reporter, or biochemical detection reporter.

In certain embodiments, the autoantibodies and tissue are autologous. In other embodiments, the autoantibodies and tissue are heterologous but are from one or more individuals of the same species.

In certain embodiments, the pre-neoplastic or neoplastic tissue is in the liver. In certain embodiments, the neoplastic tissue is a fibrolamellar hepatocellular carcinoma. In other embodiments, pre-neoplastic or neoplastic tissue is liver, skin, breast, or prostate tissue and/or cancer.

In yet another aspect, the invention provides a method of identifying pre-neoplastic or neoplastic tissue of a human comprising: (i) contacting the tissue with a labeled probe (e.g., anti-human IgG, antigen-binding fragments of anti-human IgG antibodies, Protein A, or Protein G, peptides that bind the Fc region, small molecules that bind the Fc region, or small molecules that bind human IgG and (ii) detecting the labeled probe complexed to antigen at the tissue, wherein an increase in the amount of labeled probe complexed to antigen at the tissue is indicative that the tissue is pre-neoplastic or neoplastic.

In certain embodiments, the tissue is in the body of the mammal. The mammal may be human or an animal. The tissue may be in the body of the mammal or explanted from the mammal. The detection may be done intra-operatively or prior to surgery. If the tissue is an explant, the explant may be fresh, frozen or fixed.

In certain embodiments, detection occurs by detecting an increase in the amount of autoantibodies complexed to antigen at the tissue as compared to that of a control sample.

In certain embodiments, the autoantibodies and tissue are autologous. In other embodiments, the autoantibodies and tissue are heterologous but are from one or more individuals of the same species.

In certain embodiments, the pre-neoplastic or neoplastic tissue is in the liver. In certain embodiments, the neoplastic tissue is a fibrolamellar hepatocellular carcinoma. In other embodiments, pre-neoplastic or neoplastic tissue is liver, skin, breast, or prostate tissue and/or cancer.

In yet another aspect, the invention provides a method of diagnosing prostate cancer in a subject comprising detecting autoantibodies complexed to antigen in the prostate secretions of the subject, wherein an increase in the amount of autoantibodies complexed to antigen in the secretions of the subject is indicative of prostate cancer.

In certain embodiments, the detection of autoantibodies complexed to the antigen comprises detecting an increase in the amount of autoantibodies complexed to antigen in the prostate secretion as compared to that of a control sample.

In certain embodiments, the mammal is a human or an animal. In certain embodiments, detection occurs intra-operatively. In certain embodiments, detection occurs prior to surgery.

In certain embodiments, the secretion is ejaculate. In certain embodiments, the secretion is urine.

In certain embodiments, the autoantibodies are labeled with a label. In certain embodiments, the label is an optical reporter, a positron-emission tomography reporter, a magnetic resonance imaging reporter, or a biochemical marker. In certain embodiments, the labeled antibody is labeled with a reporter that can be detected optically such as a positron emission tomography reporter, CT reporter, X-ray reporter, magnetic resonance imaging reporter, luminescence reporter, RAMAN spectroscopy reporter, surface enhanced Raman spectroscopy (SERS) reporter, second harmonic generation reporter, or biochemical detection reporter.

In certain embodiments, the autoantibodies are detected using an antibody detection reagent. In certain embodiments, the antibody detection reagent is Anti-IgG, Protein A, Protein G, an anti-human IgG, Fab(2) fragment of an anti-human IgG, Fab(1) of an anti-human IgG and a humanized mouse anti-human IgG, peptides that bind to the Fc region of human antibodies, and small molecules that bind IgGs.

In certain embodiments, the autoantibodies and sample are autologous. In other embodiments, the autoantibodies and sample are heterologous but are from one or more individuals of the same species.

In yet another aspect, the invention provides a method of killing pre-neoplastic or neoplastic tissue of a mammal comprising: (i) isolating autoantibodies from the mammal, (ii) complexing a toxin to the autoantibodies, and (iii) contacting the tissue with the toxin-autoantibodies complex, wherein said toxin-autoantibodies complex kill the pre-neoplastic or neoplastic tissue.

In certain embodiments, the toxin is paclitaxel, adriamycin, beta-emitters, or ricin.

In certain embodiments, the mammal is a human or an animal. In certain embodiments, the autoantibodies and tissue are autologous. In other embodiments, the autoantibodies and tissue are heterologous but are from one or more individuals of the same species.

In certain embodiments, the pre-neoplastic or neoplastic tissue is in the liver. In certain embodiments, the neoplastic tissue is a fibrolamellar hepatocellular carcinoma. In other embodiments, pre-neoplastic or neoplastic tissue is liver, skin, breast, or prostate tissue and/or cancer.

In yet another aspect, the invention provides a method of killing pre-neoplastic or neoplastic tissue of a mammal comprising: (i) complexing a toxin to molecules that recognize autoantibodies and (ii) contacting the tissue with the toxin-molecule conjugate, wherein said toxin-molecule-autoantibody complex kill the pre-neoplastic or neoplastic tissue.

In certain embodiments, the molecules are antibody recognition molecules such as anti-human IgG, antigen-binding fragments of anti-human IgG antibodies, Protein A, or Protein G, peptides that bind IgGs, and small molecules that bind IgGs.

In certain embodiments, the toxin is paclitaxel, adriamycin, beta-emitters, or ricin.

In certain embodiments, the mammal is a human or an animal. In certain embodiments, the autoantibodies and tissue are autologous. In other embodiments, the autoantibodies and tissue are heterologous but are from one or more individuals of the same species.

In certain embodiments, the pre-neoplastic or neoplastic tissue is in the liver. In certain embodiments, the neoplastic tissue is a fibrolamellar hepatocellular carcinoma. In other embodiments, pre-neoplastic or neoplastic tissue is liver, skin, breast, or prostate tissue and/or cancer.

### BRIEF DESCRIPTION OF THE DRAWINGS

FIG. 1. Demonstrates four mouse models of cancer probed with secondary anti-mouse IgG conjugated to Alexa Fluor 488, with a DAPI counterstain. Row (a) depicts the alb-myc model with carcinoma in the first column, adenoma in the second column, and normal liver Wild-type (“WT”) in the third column. Row (b) depicts MMTV-neu model with carcinoma in first column, hyperplasia with atypia in second column, and normal WT mammary tissue in the third column. Row (c) contains prostate models. The first column contains grade 2 carcinoma from the Pb-myc model, the second column is PIN tissue from the PTEN knockout model, and the third column has normal WT prostate tissue. Scale bars=1 mm.

FIG. 2. Bar graphs illustrate the relative intensities (mean±SEM) of (a) alb-myc liver tissue vs. WT, (b) alb-myc graded liver tissue vs. grade 0, (c) abnormal breast tissue vs. grade 0, (d) abnormal prostate tissue vs. WT, (e) alb-myc organ tissue vs. WT, (f) MMTVneu organ tissue vs. WT The y-axis is the log-fold relative intensity of test tissue to control, while the x-axis lists the various grades or types of tissue. P-values were calculated using the log-transformed ratios of the mean fluorescent intensity in the top 100 intensity values (clustered one-way random effects model). Neu and myc (e, f) represent organs with known expression of MMTV and albumin respectively.

FIG. 3. In the alb-myc mouse model for liver cancer (a) hematoxylin and eosin stain (“H&E”) is shown with (b) corresponding horse anti-mouse IgG immunofluorescence. Area circled is Grade 3, corresponding to region of immunofluorescence. In the Pb-myc mouse model for prostate cancer (c) H&E is shown with (d) corresponding horse anti-mouse IgG immunofluorescence. Area circled is region of prostatic intraepithelial neoplasia (PIN), corresponding to region of immunofluorescence in stroma and secretions (arrows).

FIG. 4. Images of transitioned tissue from mouse models (left column) with corresponding WT (right column). In the alb-myc model (a), the antibodies are binding both sinusoidal endothelial cells (arrowheads) and hepatocytes (arrows). In the MMTV-neu model (b), the antibodies are binding atypical ductal and alveolar cells (arrows), in addition to, adipocytes (arrowheads), endothelial cells, and collagen. The PTEN model (c) is showing antibody binding in the PIN cells (arrows) as well as the fibrovascular stroma of the prostate (arrowheads). Scale bar=50 μm.

FIG. 5. 4T1 tumor from immunocompetent (left column) and immunosuppressed (right column) mice probed with anti-mouse IgG (a) conjugated to Alexa Fluor 488 with a DAPI counterstain. Scale bar=200 μm. Magnified images from within tumor (b) and within microenvironment (c). Scale bar=50 μm (b,c).

FIG. 6. Shows liver, spleen, and lung from immunocompetent 4T1 xenograft mice. (a) Histologically normal sections from an immunocompetent mouse. (b) Sections from a mouse with AMH/leukemia. All tissue probed with secondary anti-mouse IgG conjugated to Alexa Fluor 488, with a DAPI counterstain. Scale bar=500 μm.

FIG. 7. Endogenous antibody identifies malignancy on both frozen section and in situ. (a,b) Frozen section immunofluorescence of breast tissue. The fluorescence from an anti-mouse IgG probe is white. (a) A mammary intraepithelial neoplasia from an MMTV-neu transgenic mouse showing extensive mouse auto-antibody staining. These results demonstrate that autoantibodies can be used to identify tumor regions in fresh frozen excised sections. (b) A healthy area of tissue with undetectable auto-antibody labeling. (c) In situ optical imaging of a 4T1 breast tumor xenograft in a balb/c mouse. Simultaneous incubation with rat anti-mouse IgG and goat anti-rabbit IgG demonstrate tumor specificity when background staining (as determined by the anti-rabbit signal) is subtracted from the anti-mouse fluorescence. This mouse had two distinct tumors designated by white asterisks. These results demonstrate that autoantibodies can be used as a tumor imaging tool during real-time intraoperative image guided surgery.

FIG. 8. Shows alb-myc liver tissue (left column) and WT liver tissue (right column). Tissue was probed (a) with goat anti-rabbit IgG conjugated to Alexa Fluor 488 with a DAPI counterstain, and (b) with DAPI counterstain only.

FIG. 9. A. Detection of Mouse IgG Antibody in a slice of liver from LEFT: a mouse that has a tumor and RIGHT a mouse that does not have a tumor. B. Enlargement of the liver on the upper left to show LEFT: Hepatocytes; CENTER: Sinusoids; RIGHT: Vessels. C. Quantification of the difference in intensity of IgG in livers from WT and tumor bearing livers shown on log (top) and linear (bottom) plots.

FIG. 10. Tumor necrosis from alb-myc liver tumor (left, exposure time 5 ms with gain 2) and leukemia tumor in the liver of a 4T1 xenograft mouse (right, exposure time 10 ms) probed with anti-mouse IgG conjugated to Alexa Fluor 488 and a DAPI counterstain. There is anti-mouse IgG staining of nuclei (double arrows), cytoplasm (arrowheads), and cell membrane (single arrows) in both models. Scale bar=25 μm.

FIG. 11. IgG from a mouse (autoantibodies) were purified, tagged with fluorophore (ICG), mixed with an anti-chicken (Igγ) labeled with a different fluorophore (Cy5.5), and both together were reinjected back into the mouse. During the first two hours on a whole animal scan, most of the fluorescence from the autoantibodies (top row) and anti-chicken antibodies (bottom row) is observed in the heart and liver (in this figure fluorescence is show as BLACK to show contrast against the white of the fur). However, from six hours onward, the autolabel is observed predominantly at the tumor, unlike the anti-chicken antibody which was never observed to accumulate at the tumor.

FIG. 12. After imaging a whole mouse (as in FIG. 11), the mouse was anaestitized, and surgically opened to reveal the tumor. The tumor showed significant fluorescence from the autoantibodies in the left figure and no detectable fluorescence from the anti-chicken antibody in the right figure (in FIG. 11 fluorescence is shown as WHITE to enhance contrast with the internal organs). When the tumor was resected, all of the fluorescence was excised from the animal (middle figure) leaving no fluorescence from auto-antibodies in the animal.

FIG. 13. A biopsy of human hepatocellular carcinoma was probed with an goat anti-human IgG antibody. Areas of human antibody are shown in white.

### DETAILED DESCRIPTION OF THE INVENTION

### 1. Overview

This invention generally relates to a method of identifying pre-neoplastic or neoplastic tissue of a mammal. In one embodiment, autoantibodies, either from the same individual or a member of the same species, are directly labeled. The labeled antibodies are then introduced into the body and the pre-neoplastic or neoplastic tissue is detected by directly detecting the labeled autoantibodies.

In another embodiment, the autoantibodies are detected indirectly by detecting autoantibodies already bound to the pre-neoplastic or neoplastic tissue. In this embodiment, the autoantibodies are detected by a detection agent.

This invention also relates to diagnosing prostrate cancer in a subject by detecting autoantibodies found in prostate secretions of the subject.

This invention also relates to a method of killing tumor cells. In an embodiment, the tumor cells are killed by directly conjugating a subject's own autoantibodies, or the autoantibodies of another subject of the same species, to a toxin and reintroducing the toxin-conjugated autoantibodies into the subject. In this embodiment, the toxin-conjugated autoantibodies bind to the neoplastic cells to kill them. In another embodiment, the toxin is conjugated to molecules which, in turn, can recognize the autoantibodies already bound to the tumor. In one embodiment the subject is a human. In another embodiment the subject is an animal.

Long-term survival in patients with various types of cancer could be greatly improved by 1) earlier detection of neoplasia; 2) greater sensitivity and specificity for detecting small foci; 3) greater sensitivity for detecting altered cells at the margins; 4) the ability to continue to detect and treat tumors even as they change. As described and exemplified herein, the instant invention relates to the discovery that polyclonal spectrum of autoantibodies bound to tissue can be used diagnostically as a means to detect microscopic foci of tumor, and distinguish transformed from normal tissue. Analysis revealed a significantly higher concentration of autoantibodies in four transgenic mouse models and two xenograft mouse models of cancer than cognate wild-type. Specifically, there was greater autoantibody binding within proliferating and transitioning tissue in all six mouse models. The increasing binding of the autoantibodies was detectable from in vitro slices of tumor and healthy tissue, from in situ tissue in a living animal during surgery and in a living animal in whole body imaging. These results indicate that the spectrum of autoantibodies bound in tissue can detect tumors at early stages of transformation. Further, they continue to track transformed cells even as the tumor undergoes mutations. This invention utilizes agents that bind autoantibodies for cancer detection and therapeutics.

As exemplified below, autoantibodies bind pre-neoplastic and neoplastic tissue in transgenic and xenograft mouse models of cancer. These autoantibodies distinguished the abnormal tissue from normal tissue within the same animal and from WT controls. Thus, the heterogeneous collection of an individual's antibodies bound to tissue is useful for detecting and targeting pre-neoplastic or neoplastic lesions. This sensitivity is the consequence of two features of the autoantibodies. First, they are diverse, recognizing a wide collection of antigens. Second, they are continuously responding to mutations in the tumor over time. Probes that recognize IgG can detect this changing spectra of antibodies. This offers the potential for improved patient outcomes by earlier detection of neoplasia, unrecognized foci and transformed cells at the margins of resection, even at the level of single cells. Additionally, the ability to target autoantibodies, which are continually tracking changes of tumors, offers therapeutic potential.

As exemplified below, the results show that early transitioning cells, are distinguishable from surrounding tissue, even before there is frank neoplasia. There were numerous regions of alb-myc livers that were histopathologically “normal”, but had high concentrations of autoantibodies. These regions also bound anti-CD34, a marker of fenestrated, capillarized endothelial cells that is not found on normal liver sinusoidal cells (Scoazec and Feldmann, 1991). This suggests that autoantibodies are recognizing transitioning areas not detectable by histopathology. In the MMTV-neu model, autoantibodies bound to atypical hyperplastic glands were observed, discriminating between this pre-neoplastic tissue and surrounding normal tissue. Thus, the autoantibodies can recognize early foci of transformation.

In all models tested, the autoantibodies bound the tumor microenvironment. The tumor has been described as an “ecosystem”: neoplastic cells combined with a milieu that aids in their growth (Bissell and Radisky, 2001). The microenvironment is known to be made up of cells, including fibroblasts, immune cells, and endothelial cells, that are activated or recruited by the nearby tumor cells to aid in the sustainment and growth of the tumor itself. The communication between this reactive microenvironment and the cancer cells affects the phenotype of the tumor (Mueller and Fusenig, 2004). Disrupting this microenvironment has a detrimental effect on tumors (Mueller and Fusenig, 2004; Roskelley and Bissell, 2002). The tumor microenvironment contains a variety of immune cells including neutrophils, dendritic cells, macrophages, and lymphocytes (Coussens and Werb, 2002).

The results of the instant invention show that autoantibodies are also present in the tumor microenvironment. In the alb-myc model, antibodies bound to the hepatocytes, but also to the liver sinusoidal endothelial cells. Alterations in these cells have been reported in early stages of liver tumorigenesis (Frachon et al., 2001). In the MMTV-neu model, antibody bound to the mammary adipocytes, endothelial cells and connective tissue stroma. Stroma adjacent to mammary tumor undergoes both phenotypic and epigenetic transformations (Fiegl et al., 2006; Hu et al., 2005; Trimboli et al., 2009). Mammary endothelial cells have been reported to be involved with tumor survival, proliferation, and invasiveness (Franses et al., 2011). Furthermore, mammary cancer-associated fibroblasts have been shown to promote tumor growth. (Orimo et al., 2005; Tyan et al., 2011) Similar results were observed in the prostate models, with antibody bound throughout the microenvironment, an altered area supporting the growth of the surrounding prostate tumor (Chung et al., 2005; Dakhova et al., 2009). Autoantibodies were also observed in the secretions of the prostate.

The results below demonstrate autoantibody binding is the result of a tumor-specific immune response. First, in the mouse models for cancer only anti-mouse IgG antibodies bound tissue, antibodies against IgG of other species or antibodies against other immunoglobins did not bind. Second, the areas to which they bound were independently shown to either be neoplastically transformed (as assayed by H&E) or transitioning (as assayed by binding of anti-CD34). Third, the antibody binding was specific to the organs generating the tumor. For example, when comparing MMTV-neu to WT mice, the mammary gland is the only organ that has a higher autoantibody binding. Moreover, in the xenograft model, the antibody response was specific to where the tumor was implanted. There were two exceptions to this observation. In the alb-myc model, autoantibodies were found bound to organs other than the liver. However, these organs also express the albumin promoter, which was used to drive the expression of the myc oncogene. Additionally, in the xenograft model, one mouse had autoantibody within the liver, spleen, and lung. On H&E, these organs were found to contain spontaneous AMH/leukemia. These results seen in a spontaneous hematopoietic cancer are consistent with the genetically engineered or xenograft solid tumor model results. During whole animal imaging of MMTV-neu mice, antibody binding was found in one mouse extraneous to the breast and, upon autopsy, this was found to be a spontaneous tumor.

### 2. Definitions

As used herein, the singular forms “a,” “an,” and “the” include plural references unless the content clearly dictates otherwise.

The term “about,” as used here, refers to +/−10% of a value.

An “antigen” refers to a molecule containing one or more epitopes (either linear, conformational or both) that elicit an immunological response. The term may be used interchangeably with the term “immunogen.” An “epitope” is that portion of given species (e.g., an antigenic molecule or antigenic complex) that determines its immunological specificity. An epitope is within the scope of the present definition of antigen. Commonly, an epitope is a polypeptide or polysaccharide or a folded domain in a naturally occurring antigen. In artificial antigens it can be a low molecular weight substance such as an arsanilic acid derivative. Normally, a B-cell epitope will include at least about 5 amino acids but can be as small as 3-4 amino acids. A T-cell epitope, such as a CTL epitope, will typically include at least about 7-9 amino acids, and a helper T-cell epitope will typically include at least about 12-20 amino acids. The term “antigen” denotes both subunit antigens, i.e., antigens which are separate and discrete from a whole organism or cell with which the antigen is associated in nature, or tumor cells, such as tumor antigens.

The term “adjuvant” or “immunological adjuvant” refers to any substance that assists or modifies the action of an antigen in the immune system. Adjuvants can potentiate humoral and/or cellular immunity.

The term “antibody” refers to an immunoglobulin or antigen-binding fragment thereof, and encompasses any such polypeptide comprising an antigen-binding fragment of an antibody. The term includes but is not limited to polyclonal, monoclonal, monospecific, polyspecific, humanized, human, single-chain, single-domain, chimeric, synthetic, recombinant, hybrid, mutated, grafted, and in vitro generated antibodies. The term “antibody” also includes antigen-binding fragments of an antibody. Examples of antigen-binding fragments include, but are not limited to, Fab fragments (consisting of the V_{L}, V_{H}, C_{L} and C_{H}1 domains); Fd fragments (consisting of the V_{H} and C_{H}1 domains); Fv fragments (referring to a dimer of one heavy and one light chain variable domain in tight, non-covalent association); dAb fragments (consisting of a V_{H} domain); single domain fragments (V_{H} domain, V_{L} domain, V_{HH} domain, or V_{NAR} domain); isolated CDR regions; (Fab′)₂ fragments, bivalent fragments (comprising two Fab fragments linked by a disulphide bridge at the hinge region), scFv (referring to a fusion of the V_{L} and V_{H} domains, linked together with a short linker), and other antibody fragments that retain antigen-binding function.

The term “autoantibody” as used herein refers to an antibody that recognizes the cells, tissues, native proteins, or molecules of the organism in which it was formed. Autoantibodies can be from an organism or individual that is heterologous to the tissue in which the detection or killing is occurring, but are from one or more individuals of the same species. The production of autoantibodies in cancer is the result of an autoimmune response directed to molecules that are overexpressed, mutated, or aberrantly regulated or localized as a result of cellular transformation of the tissue to a cancerous, neoplastic, pre-cancerous or pre-neoplastic phenotype. These molecules include, but are not limited to proteins, lipids, glycolipids, and nucleic acids.

The term “neoplastic tissue,” “neoplastic cells,” or “neoplasms” as used herein refers to an abnormal mass of tissue or a proliferation of cells. The growth of neoplastic cells exceeds that of normal tissue around it and it is not coordinated with that of the normal tissue around it. Neoplasms may be benign, pre-malignant (e.g., carcinoma in situ) or malignant (e.g., cancer). This tissue can originate from any cell type or tissue found in a mammal, including, but not limited to hepatic, skin, breast, prostate, neural, optic, intestinal, cardiac, vasculature, lymph, spleen, renal, bladder, lung, muscle, connective, tissue, pancreatic, pituitary, endocrine, reproductive organs, bone, and blood. More particularly, the neoplastic tissue is from the liver, skin, breast, prostate, and lymph. More particularly, the neoplastic tissue is from the liver. Even more specifically, the neoplastic tissue is a fibrolamellar hepatocellular carcinoma.

The term “pre-neoplastic tissue” as used herein refers to tissue preceding the formation of a benign or malignant neoplasm. This tissue can originate from any tissue found in a mammal, including, but not limited to liver, skin, breast, prostate, neural, optic, intestinal, cardiac, vasculature, lymph, spleen, renal, bladder, lung, muscle, connective, tissue, pancreatic, pituitary, endocrine, reproductive organs, bone, and blood. More particularly, the neoplastic tissue is from the liver, skin, breast, prostate, and lymph. More particularly, the neoplastic tissue is from the liver. Even more specifically, the neoplastic tissue is a fibrolamellar hepatocellular carcinoma.

The term “hyperplasia” as used herein refers to an increase in number of cells/proliferation of cells. It may result in the gross enlargement of an organ. Hyperplasia is a common pre-neoplastic response to a stimulus.

The term “fragment” as used herein refers to a physically contiguous portion of the primary structure of a biomolecule. In the case of polypeptides, a fragment may be defined by a contiguous portion of the amino acid sequence of that protein and may be at least 3-5 amino acids, at least 6-10 amino acids, at least 11-15 amino acids, at least 16-24 amino acids, at least 25-30 amino acids, and at least 30-45 amino acids. In the case of polynucleotide, a fragment is defined by a contiguous portion of the nucleic acid sequence of that polynucleotide and may be at least 9-15 nucleotides, at least 15-30 nucleotides, at least 31-45 nucleotides, at least 46-74 nucleotides, at least 75-90 nucleotides, and at least 90-130 nucleotides. In some embodiments, fragments of biomolecules are immunogenic fragments.

The term “intra-operatively” as used herein refers to occurring, carried out, or encountered in the course of surgery.

The term “autologous” as used herein refers to a situation in which the donor and recipient are the same individual.

The term “heterologous” as used herein refers to a situation in which the donor and recipient are from different individuals. The different individuals can be of the same species or different species.

The term “control sample” as used herein refers to a background signal or a signal associated with a normal tissue (i.e., a tissue that is not pre-neoplastic or neoplastic). The control sample can be autologous, from the same animal, or heterologous, from another animal. The control sample can be from the same species or different species.

The term “secretion” as used herein refers to a substance, such as saliva, fluid, mucus, tears, bile, or a hormone, that is secreted from a cell or gland.

The term “complexing” or “complex” as used herein refers to the binding or association of an antibody or autoantibody to an antigen, cell, or tissue. It can also mean the binding or association of a molecule, such as a reporter or toxin, to an autoantibody or the binding or association of a molecule, such as a reporter or toxin, to a different molecule that can recognizes an autoantibody.

### 3. Identifying Pre-Neoplastic and Neoplastic Tissues Intra-Operatively or from a Biopsy

While various groups have identified a number of tumor-associated autoantibodies in the hopes of utilizing them as biomarkers, prognostic factors, and indicators of tumor recurrence, they have only looked in the sera of patients. One embodiment of the present invention, however, focuses on autoantibodies as they are associated with the pre-neoplastic or neoplastic tissue itself. By allowing for identification and imaging of the tissue itself, this invention allows for the detection of both pre-neoplastic and neoplastic tissue, not just the presence of the autoantibody within the sera. Thus, this invention leads to improved staging of disease. Earlier diagnosis of tumor recurrence, the ability to detect small foci that can not be detected optically as well as detect altered cells at the margins of a resection. This detection can occur in a biopsy sample, a secretion sample, in situ (such as intra-operatively or intravital staining), or a whole body scan.

Pre-neoplastic and neoplastic tissue can be detected on biopsy samples of tissue or secretion samples removed for examination prior to surgical intervention. Types of biopsies used in relation to this invention include, but are not limited to needle biopsy, CT-guided biopsy, ultrasound-guided biopsy, liver biopsy, bone biopsy, bone marrow biopsy, liver biopsy, kidney biopsy, aspiration biopsy, prostate biopsy, skin biopsy. Secretion samples can be collected by ejaculation, urination, voiding, swabbing, collecting, scraping, or needle aspiration of the secretion.

The instant invention provides a new strategy for identifying pre-neoplastic or neoplastic tissue. In particular, autoantibodies can be used to distinguish transitioned tissue from normal tissue intra-operatively in two ways. First, abnormal tissue can be detected in freshly excised or frozen sections of tissue slides. Frozen sections are commonly used for real-time analysis of margins of surgical specimens. Further resection or enhanced treatment regimens are often employed based on these results. However, the sensitivity of frozen sections recognizing positive surgical margins is inconsistent. For example, the sensitivity of frozen section analysis for positive surgical margins during radical prostatectomy for prostate cancer is 42% (Tsuboi et al., 2005). Supplementing or supplanting current pathological frozen section analysis with the present invention increases this sensitivity leading to a more precise intra-operative test.

Second, autoantibodies can detect abnormal tissue in situ during surgical resection of tumor. Therefore, autoantibodies can be applied to intraoperative imaging to assess for the presence of neoplasia on the cellular level. The use of near-infrared fluorescent probes to detect autoantibodies can be combined with the recent development of a real-time intraoperative imaging device (Liu et al., 2011) and other inter-operative technologies (Liu et al., 2011; Ye et al., 2011). The presence of tagged autoantibodies within transitioned tissue will also allow for a more accurate resection of all diseased tissue.

The detection can occur by one of two methods. The first method comprises the labeling of the autoantibodies, wherein the labeled autoantibodies are used to detect the pre-neoplastic or neoplastic tissue by introducing the labeled autoantibodies into the subject. The second method comprises the detection of autoantibodies indirectly by introducing an autoantibody detection agent.

In one embodiment the labeled autoantibodies or the autoantibodies detected by the reagent are bound to the pre-neoplastic or neoplastic tissue. In another embodiment, the labeled autoantibodies or the autoantibodies detected by the reagent are bound to individual pre-neoplastic or neoplastic cells. In yet another embodiment, the labeled autoantibodies or the autoantibodies detected by the reagent are bound to antigens on the pre-neoplastic or neoplastic cells. In another embodiment, the antigen is an antigen known to be expressed by a cancerous cell.

In one embodiment, the pre-neoplastic or neoplastic tissue and cells are derived from bone, blood, hepatic, skin, breast, prostate, neural, optic, intestinal, cardiac, vasculature, lymph, spleen, renal, bladder, lung, muscle, connective, pancreatic, pituitary, endocrine, or reproductive organ tissue. In one embodiment, the neoplastic tissue or neoplastic cells are cancerous. In yet another embodiment, the type of cancer is bone, blood, or hepatic, skin, breast, prostate, neural, optic, intestinal, cardiac, vasculature, lymph, spleen, renal, bladder, lung, muscle, connective, pancreatic, pituitary, endocrine, or reproductive organ tissue cancers. In another embodiment, the pre-neoplastic or neoplastic tissue is liver, breast, skin, or prostate tissue. In another embodiment, the neoplastic tissue is liver, breast, skin, or prostate cancer. In another embodiment, the cancer is fibrolamellar hepatocellular carcinoma. In another embodiment, the cancer is leukemia, adrenocarcinoma, astocytomeas, basal cell carcinoma, osteosarcoma, glioma, chordoma, retinoblastoma, squalors cancer, or T-cell lymphoma.

a. Detection of Labeled Autoantibodies

In one embodiment, autoantibodies are directly labeled. In another embodiment the autoantibodies are isolated before being labeled. In yet another embodiment the autoantibodies are isolated and purified before being labeled. The labeled antibodies are then introduced or introduced or reintroduced back into the subject and the pre-neoplastic or neoplastic tissue is detected by measuring an increase in the amount of autoantibodies complexed to the tissue, cell, or antigen. In one embodiment, the detection of autoantibodies complexed to an antigen comprises detecting an increase in the amount of autoantibodies complexed to antigen at the tissue as compared to that of a control sample.

In one embodiment, an increase in signal above control or background of at least 2% indicates the detection of a pre-neoplastic tissue. In another embodiment, an increase in signal above control or background of at 5% indicates the detection of a pre-neoplastic tissue. In another embodiment, an increase in signal above control or background of at least 10% indicates the detection of a pre-neoplastic tissue. In another embodiment, an increase in signal above control or background of at least 15, 20, 25, 30, 40, 50, 60, 70, 80, 90, 100, 150, 200, or 250% indicates the detection of a pre-neoplastic tissue.

In one embodiment, an increase in signal above control or background of at least 2% indicates the detection of a neoplastic tissue. In another embodiment, an increase in signal above control or background of at 5% indicates the detection of a neoplastic tissue. In another embodiment, an increase in signal above control or background of at least 10% indicates the detection of a neoplastic tissue. In another embodiment, an increase in signal above control or background of at least 15, 20, 25, 30, 40, 50, 60, 70, 80, 90, 100, 150, 200, or 250% indicates the detection of a neoplastic tissue.

In one embodiment, the autoantibodies are taken from the subject being tested for the pre-neoplastic or neoplastic tissue (i.e., the autoantibodies and the tissue are autologous). In this embodiment, the labeled autoantibodies are reintroduced back into the subject and the pre-neoplastic or neoplastic tissue is detected by measuring an increase in the amount of autoantibodies complexed to the tissue, cell, or antigen.

In another embodiment, the autoantibodies and tissue are heterologous but from the same species. In yet another embodiment, the autoantibodies and tissue are heterologous but from different species. In this embodiment, the labeled autoantibodies are introduced into the subject and the pre-neoplastic or neoplastic tissue is detected by measuring an increase in the amount of autoantibodies complexed to the tissue, cell, or antigen. In yet another embodiment, the heterologous autoantibodies are pooled from multiple individuals.

Methods for isolating autoantibodies are well known in the art. See, for example, Current Protocols in Immunology, Cooligan, et al. (eds.), National Institutes of Health, John Wiley and Sons, Inc., 1995; Sambrook et al., Molecular Cloning: A Laboratory Manual, Second Edition, Cold Spring Harbor, N.Y., 1989; and Hurrell, J. G. R., Ed., Monoclonal Hybridoma Antibodies: Techniques and Applications, CRC Press, Inc., Boca Raton, Fla., 1982. A number of companies make kits, along with instructions on their implementation: www.piercenet.com/browse.cfm?fldID=ACC3CB7A-1097-403A-BAC1-E6F8AAD12044.

Labeling or tagging of the autoantibodies of the present invention is done according to methods of antibody generally known in the art. Methods for labeling antibodies are well known in the art. Examples of labeling substances include, but are not limited to, an optical reporter [such as bioluminescence (e.g. luciferase), near-infrared and visible fluorescent labels, fluorophores/fluorochromes (e.g., Hydroxycoumarin, Aminocoumarin, Methoxycoumarin, Cascade Blue, Pacific Blue, Pacific Orange, Lucifer Yellow, NBD, R-Phycoerythrin, PE-Cy5 conjugates, PE-Cy7 conjugates, Red 613, PerCP, TruRed, Fluor X, Fluorescein, Indocyanine Green (ICG), BODIPY-FL, TRITC, X-Rhodamine, Lissamine Rhodamikne B, Texas Red, fluorescent proteins (e.g. CFP, GFP, YPF, mCherry, mPlum, mStrawberry, and RFP and photoconvertable fluorescent proteins), Cy dyes (e.g., Cyanine, Cy3, Cy5, Cy5.5, Cy7), Alexa Fluor dyes, Atto Dyes, Dylight, IRIS Dyes, Seta dyes, SeTau dyes, SRfluor dyes and Square dyes)], a positron-emission tomography reporter [such as carbon, iodine, nitrogen, oxygen, fluoride, and zirconium isotopes (e.g., carbon-11, nitrogen-14, oxygen-15, fluorine-18, iodine-124, zirconium-89, and copper-64), fludeoxyglucose, and 11C-metomidate], a magnetic resonance imaging reporter [such as iron oxide agents (e.g., Cliavist, Combidex, Endorem, Feridex, Resovist, Sinerem), boron, magnesium chelators, and gadolinium agents (e.g., Omniscan, Multihance, Magnevist, ProHance, Vasovist, Eovist and OptiMARK, gadocoletic acid gadodenterate, gadomelitol, and gadopenamide)], and a biochemical marker [such as proteins (e.g., biotin, avidin, neutravidine, photobiotin, strep-tag, streptavidin) and enzymes (e.g., horseradish peroxidase, alkali phosphatase)].

The labeled autoantibodies can be detected by light/optical microscopy with a fluorescence microscope (e.g., confocal microscopy, multiphoton microscopy, whole animal fluorescence imaging and stimulated emission depletion microscopy), by positron emission tomography (PET) scans, magnetic resonance imaging (MRI) scans, or biochemical assays (e.g., ELISA, radioimmunoassay, magnetic immunoassays, and reverse phase protein lysate microarray).

b. Detecting Autoantibodies Using an Antibody Detection Reagent

Detecting an autoantibody using an antibody detection reagent is well known in the art. Such reagents for antibody detection include, but are not limited to an antibody made to IgG (anti-IgG), Protein A, Protein G, Fab(2) fragment of an anti-IgG, Fab(1) of an anti-IgG, peptides that bind to the Fc region, and small molecules that bind antibodies. In one embodiment, the anti-IgG is an anti-human IgG or a humanized mouse anti-human IgG In one embodiment the anti-IgG includes the affinity ligand secretory component (Brandtzaeg, 1983), peptides that are designed based on hydropathy (Fassina et al., 1992), fragments of the placental alkaline phosphatase that can bind the Fc region (Makiya and Stigbrand, 1992), multimeric peptides (Verdoliva et al., 1995), one of the peptides from the E. coli surface exposed EiB proteins than bind IgG (Sandt and Hill, 2001), variations of the Fc receptor proteins that can bind IgG (Akilesh et al., 2007; Fridman, 1991; Fridman et al., 1984), anyone of a number of protein G or protein A mimetic (PAM) identified from peptide libraries that bind IgG (Fassina, 2000; Fassina et al., 1996; Fassina et al., 1998), or identified from combinatorial chemical synthesis (Fassina et al., 2001; Kabir, 2002), or combinatorial libraries (Nielsen et al., 2010), phage display libraries (Sakamoto et al., 2009), or rationally designed non-peptidyl mimetics of Protein A (Li et al., 1998), hexamer peptide affinity resins that bind the Fc region of IgG (Yang et al., 2005, 2009a) or the specific hexamer HWRGWV (Yang et al., 2010), use of the all-D amino acid peptide ligands (D'Agostino et al., 2008; Verdoliva et al., 2002), synthetic ligands including cyclic peptides (Verdoliva et al., 2005), a synthetic triazine scaffold substituted with 3-mainopheno and 4-amino-1-maphthol (Teng et al., 2000) or through other combinatorial chemical syntheses to make IgG binding ligands (Teng et al., 1999), affinity ligands that mimic Protein L (Roque et al., 2005), trisubstituted purine derivatives as protein A mimetics (Zacharie et al., 2010; Zacharie et al., 2009), or dendrimeric peptides (Moiani et al., 2009).

In one embodiment the reagents for detection of autoantibodies are selected from the collection of molecules that bind with high affinity to the autoantibodies in a one-to-one manner.

In one embodiment the reagents for detection of autoantibodies are selected from the collection of molecules that bind with relatively lower affinity to the autoantibodies. In this embodiment, multiple copies of the reagents for detection will be conjugated together. Thus, the lower affinity will be compensated by an increased avidity of binding to the autoantibodies.

In the case of whole animal imaging, the labeled antibody detection agent will be injected into the circulation and allowed to distribute, and then a whole body scan will be done later, such as 6 to 24 hours later. The time period for distribution will vary depending upon the antibody detection agent; for example, some equilibrate in 30 minutes some in 6 hours. In one embodiment of intraoperative imaging, the antibody detection kit is made of one component, directly targeted to the autoantibodies, that is injected into the circulation and allowed to distribute prior to surgery. In another embodiment of intraoperative imaging, the antibody detection kit is injected during surgery directly into the circulation perfusing the diseased organ. In another embodiment of intraoperative imaging the antibody detection kit will be made of two components: An agent targeted to the antibody conjugated to one fluorophore and a chemically similar agent that targets antibodies of a different species labeled with a second fluorophore. The two will be mixed together in a buffer, poured over the region of interest and then rinsed with just the buffer.

The reagents for detection of autoantibodies will be conjugated to labels or reporters that can be detected. Examples of labeling substances include, but are not limited to, an optical reporter [such as bioluminescence (e.g. luciferase), infrared, near-infrared and visible fluorescent labels, fluorophores/fluorochromes (e.g., Hydroxycoumarin, Aminocoumarin, Methoxycoumarin, Cascade Blue, Pacific Blue, Pacific Orange, Lucifer Yellow, NBD, R-Phycoerythrin, PE-Cy5 conjugates, PE-Cy7 conjugates, Red 613, PerCP, TruRed, Fluor X, Fluorescein, Indocyanine Green (ICG), BODIPY-FL, TRITC, X-Rhodamine, Lissamine Rhodamikne B, Texas Red, fluorescent proteins (e.g. CFP, GFP, YPF, mCherry, mPlum, mStrawberry, and RFP and photoconvertable fluorescent proteins), Cy dyes (e.g., Cyanine, Cy3, Cy5, Cy5.5, Cy7), Alexa Fluor dyes, Atto Dyes, Dylight, IRIS Dyes, Seta dyes, SeTau dyes, SRfluor dyes and Square dyes)], a positron-emission tomography reporter [such as carbon, iodine, nitrogen, oxygen, fluoride, and zirconium isotopes (e.g., carbon-11, nitrogen-14, oxygen-15, fluorine-18, iodine-124, zirconium-89, and copper-64), fludeoxyglucose, and 11C-metomidate], a magnetic resonance imaging reporter [such as iron oxide agents (e.g., Cliavist, Combidex, Endorem, Feridex, Resovist, Sinerem), boron, magnesium chelators, and gadolinium agents (e.g., Omniscan, Multihance, Magnevist, ProHance, Vasovist, Eovist and OptiMARK, gadocoletic acid gadodenterate, gadomelitol, and gadopenamide)], and a biochemical marker [such as proteins (e.g., biotin, avidin, neutravidine, photobiotin, strep-tag, streptavidin) and enzymes (e.g., horseradish peroxidase, alkali phosphatase)]. Thus, the labeled autoantibodies can be detected by light/optical microscopy with a fluorescence microscope (e.g., confocal microscopy, multiphon microscopy, whole animal fluorescence imaging microscopy, and stimulated emission depletion microscopy), by positron emission tomography (PET) scans, magnetic resonance imaging (MRI) scans, or biochemical assays (e.g., ELISA, radioimmunoassay, magnetic immunoassays, and reverse phase protein lysate microarray).

c. Detection in Frozen Sections

Tissue samples are obtained from surgically-removed tissue that has been frozen in a fixed or unfixed state and sectioned. The tissue can be sectioned by any means known in the art. For example, the tissue can be placed in a cryoprotective embedding medium (e.g., Tissue Tek O.C.T., TBS or Cryogel), and then the tissue sample can be snap frozen in isopentane cooled by liquid nitrogen. Tissue is then sectioned in a freezing microtome or cryostat.

Examples for suitable tissue fixatives include, but are not limited to, crosslinking fixatives (e.g., paraformaldehyde, formalin, and gluteraldehyde), precipitating fixatives (e.g., ethanol, methanol, acetone, and an alcohol in combination with acetic acid), oxidizing agents (e.g., osmium tetoxide, potassium dichromate, chromic acid, and potassium permanganate), mercurials (e.g., B-5 and Zenker's), picrates, HOPE Fixative, or other standard histological preservatives.

d. Detecting in Fresh Explants

Tissue samples are obtained from surgically-removed or biopsied tissue in a fixed or unfixed state.

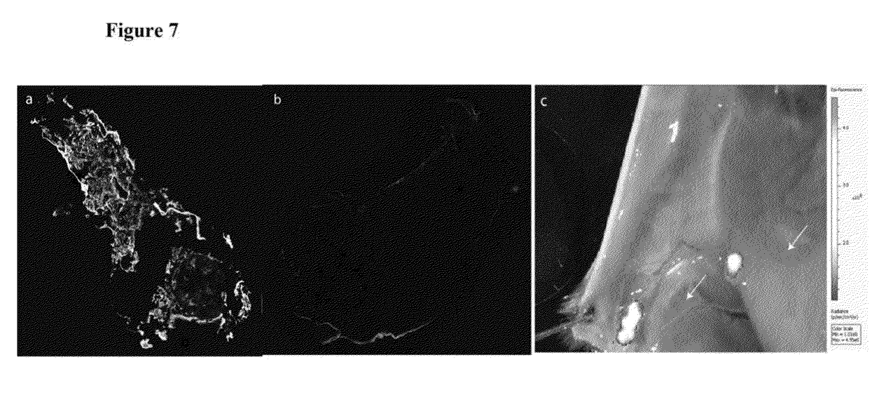

In one embodiment the tissue will be excised and then the explant will be incubated with an anti host-IgG, conjugated to one reporter and an anti-non-host IgG conjugated to a different reporter. For example, if the sample is from a human than the explanted sample will be simultaneously incubated with a humanized-mouse anti human IgG conjugated to Cy5.5 and a humanized-mouse anti-chicken IgG conjugated to ICG (indocyanine green). The fluorescence of the ICG and the Cy5.5 will be measured simultaneously and transformed cells will be assayed for by the ratio of the fluorescence of Cy5.5:ICG. The fluorescence can be quantified either by fluorescence microscopy or by a spectrofluorimeter. For the examples show (FIG. 7 a,b) the fluorescence was monitored by epi-fluorescence microscopy.

In another embodiment the tissue will be excised and then the explant will be incubated with labeled autoantibodies from the patient and then the fluorescence monitored by fluorescence microscopy. Fluorescence microscopy is a preferred method of assaying because it helps distinguish tumor for healthy regions.

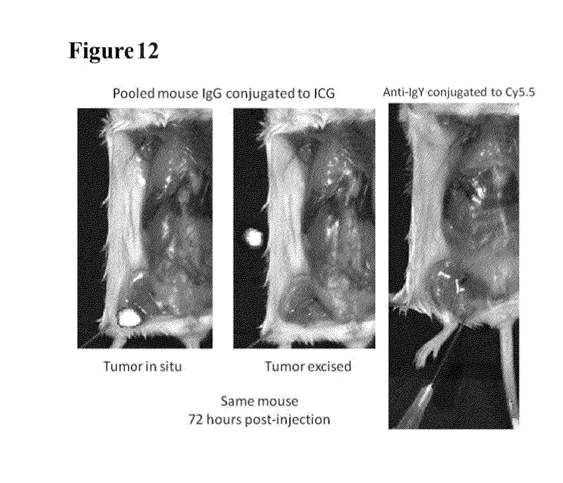

In another embodiment the animal will be injected with an anti host-IgG, conjugated to one reporter and an anti-non-host IgG conjugated to a different reporter. For example, if the host is a human than the host will be simultaneously incubated with a humanize-mouse anti-human IgG conjugated to Cy5.5 and a humanized-mouse anti-chicken IgG conjugated to ICG (indocyanine green). The antibodies will be allowed to circulate prior to surgery. Then, during surgery the sample will be removed and then imaged by fluorescence microscopy, as shown in FIG. 12. In a variation on this implementation the probes will be injected into a blood vessel feeding the diseased organ.

In another embodiment the animal will be injected with labeled autoantibodies which will be allowed to circulate prior to surgery. Then, during surgery the sample will be removed and then imaged by fluorescence microscopy, as shown in FIG. 12. In a variation on this implementation the probes will be injected into a blood vessel feeding the diseased organ. Then, during surgery the sample will be removed and then imaged by fluorescence microscopy, as shown in FIG. 12. In a variation on this implementation the labeled autoantibodies will be injected into a blood vessel feeding the diseased organ.

e. Detection Interoperative

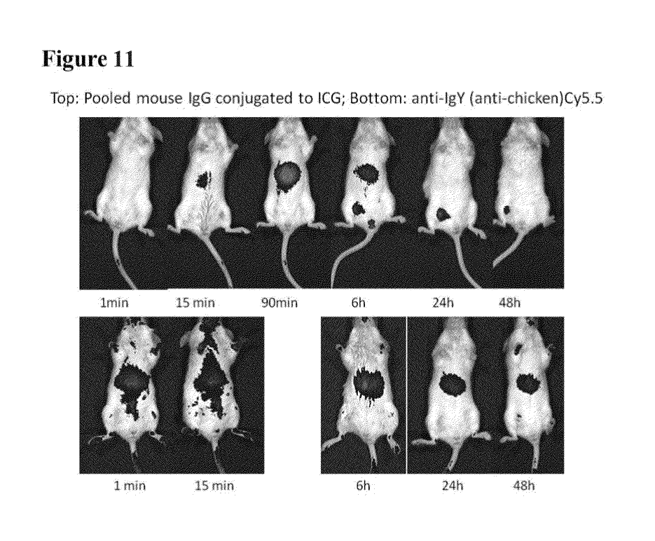

In one embodiment the animal will be injected with autoantibodies that are conjugated to a probe that can be detected by fluorescence, PET or MRI. The antibodies will be allowed to circulate prior to imaging as in FIG. 11. In a variation on this implementation the labeled autoantibodies will be injected into a blood vessel feeding the diseased organ.

In another embodiment the animal will be injected with an anti host-IgG, conjugated to a probe that can be detected by PET or MRI. The antibodies will be allowed to circulate prior to imaging. In a variation on this implementation the labeled anti-host IgG will be injected into a blood vessel feeding the diseased organ.

f. Detection in Whole Body Scans.

Detection of an antibody in a whole animal is well established by a variety of means including detection by PET, MRI and whole body fluorescence. These techniques have been used to track specific antibodies, such as Trastuzumab (trade name heurceptin). In one embodiment of this invention, the whole IgG from the mammal will be labeled as described above, and then the labeled autoantibodies introduced back into the mammal and then the mammal will be scanned in a whole body scan as described above (e.g. a PET imager, MRI, whole body fluorescence imager) as seen in FIG. 11.

In one embodiment of this invention, the autoantibodies that are already bound at the tumor will detected by labeling an agent that can detect the autoantibodies as described above. These agents will be injected into the animal and then detected (by PET, MRI, whole animal imaging as described above).

### 4. Diagnosing Prostate Cancer

The most common tests for prostate cancer in its earlier states is with regular digital prostate exams and the prostate specific antigen (PSA) blood tests. PSA is a specific type of protein whose blood level tends to increase in the presence of prostate cancer. Currently 20% of men test positive for PSA, which frequently results in a biopsy. However, most of these are false positives. A test that is more accurate than PSA (fewer false positives with no increase in false negatives) could decrease unnecessary expense and discomfort. A prostate ultrasound and biopsy are both used to evaluate the abnormal results obtained in the digital rectal exam or an elevated PSA test.

With the biopsy, transrectal ultrasound imaging is used to guide several small needles through the rectum wall into areas of the prostate where abnormalities are detected. The needles remove a tiny amount of tissue. Usually six or more biopsies are taken to test various areas of the prostate. Prostate biopsy can lead to harmful side effects such as prolonged or heavy bleeding, pain, swelling, difficulty urinating, fever, discharge from the penis, and erectile dysfunction. A prostate biopsy can also result in spreading cancer cells to other parts of the body and may also be the reason that men have a recurrence of disease many years after the prostate was removed. Additionally, it is an invasive and costly procedure. The present invention, however, is a non-invasive way to diagnose prostate cancer.

In one embodiment, diagnosing prostate cancer in a human subject comprises detecting autoantibodies complexed to antigen in the prostate secretions of the subject, wherein an increase in the amount of autoantibodies complexed to antigen in the secretions of the subject is indicative of prostate cancer. Such a test is non-invasive and does not require a biopsy. In another embodiment, the detection of autoantibodies complexed to the antigen comprises detecting an increase in the amount of autoantibodies complexed to antigen at the tissue as compared to that of a control sample.

In one embodiment, the autoantibodies are labeled autoantibodies. In another embodiment, the autoantibodies are detected using an antibody detection reagent.

In one embodiment, the secretion sample is tested for autoantibodies endogenously present in the sample by using an autoantibody detection reagent. In another embodiment, the secretion sample is contacted with autoantibodies, which are in turn detected with an autoantibody detection reagent to detect pre-neoplastic or neoplastic tissue. In another embodiment, the secretion sample is contacted with autoantibodies which are in turn detected with an autoantibody detection reagent to detect pre-neoplastic or neoplastic cells. In another embodiment, the secretion sample is contacted with autoantibodies which are in turn detected with an autoantibody detection reagent to detect pre-neoplastic or neoplastic antigen associated with prostate cancer. In one embodiment, the autoantibody is autologous. In another embodiment, the autoantibody is heterologous.

In the case of the prostate, the secretions are believed to not be pre-neoplastic nor neoplastic. Instead, they are normally never exposed to the immune system. It is only upon neoplasia that the basal layer of cells that surround the secretions are compromised, allowing the secretions to be exposed to the immune system, that you get autoantibodies to the secretions. Thus, there are autoantibodies to the secretions not because they neoplasia but because the neoplasia has compromised the barrier between the immune system and the secretions. The immune system reacts to these secretions, as it is normally naive to these secretions.

In another embodiment, the secretion sample is contacted with labeled autoantibodies to detect pre-neoplastic or neoplastic antigen. In another embodiment, the secretion sample is contacted with labeled autoantibodies to detect pre-neoplastic or neoplastic tissue. In another embodiment, the secretion sample is contacted with labeled autoantibodies to detect pre-neoplastic or neoplastic cells. In another embodiment, the secretion sample is contacted with labeled autoantibodies to detect an antigen associated with prostate cancer. In one embodiment, the labeled autoantibody is autologous. In another embodiment, the labeled autoantibody is heterologous.

In one embodiment, the labeled autoantibodies or the autoantibodies detected by the reagent are bound to the pre-neoplastic or neoplastic tissue. In another embodiment, the labeled autoantibodies or the autoantibodies detected by the reagent are bound to individual pre-neoplastic or neoplastic cells. In yet another embodiment, the labeled autoantibodies or the autoantibodies detected by the reagent are bound to antigens on the pre-neoplastic or neoplastic cells.

In one embodiment, an increase in signal above control or background of at least 2% indicates the detection of a pre-neoplastic tissue. In another embodiment, an increase in signal above control or background of at 5% indicates the detection of a pre-neoplastic tissue. In another embodiment, an increase in signal above control or background of at least 10% indicates the detection of a pre-neoplastic tissue. In another embodiment, an increase in signal above control or background of at least 15, 20, 25, 30, 40, 50, 60, 70, 80, 90, 100, 150, 200, or 250% indicates the detection of a pre-neoplastic tissue.

In one embodiment, an increase in signal above control or background of at least 2% indicates the detection of a neoplastic tissue. In another embodiment, an increase in signal above control or background of at 5% indicates the detection of a neoplastic tissue. In another embodiment, an increase in signal above control or background of at least 10% indicates the detection of a neoplastic tissue. In another embodiment, an increase in signal above control or background of at least 15, 20, 25, 30, 40, 50, 60, 70, 80, 90, 100, 150, 200, or 250% indicates the detection of a neoplastic tissue.

### 5. Targeting and Killing Pre-Neoplastic and Neoplastic Tissues

The ability to specifically target the autoantibodies and their bound target in the tumor offers a potential therapeutic target for stalling, senescing, killing, or eradicating pre-neoplastic and neoplastic tissue.

In one embodiment, autoantibodies are isolated (i.e., by purification) from the subject and complexed with toxin. The toxin-autoantibody complex is then introduced to the subject, wherein said toxin-autoantibodies complex kill the pre-neoplastic or neoplastic tissue.

In one embodiment, a sample containing autoantibodies is taken from the subject and the autoantibodies are complexed with toxin. The toxin-autoantibody complex is then reintroduced back into the subject harboring the pre-neoplastic or neoplastic tissue or cells.

In one embodiment, autoantibodies are isolated from the subject and complexed with toxin. The toxin-autoantibody complex is then introduced into the general circulation of the subject, wherein said toxin-autoantibodies complex kill the pre-neoplastic or neoplastic tissue.

In one embodiment, autoantibodies are isolated from the subject and complexed with toxin. The toxin-autoantibody complex is then introduced into the blood vessels that perfuse the organ(s) containing the pre-neoplastic or neoplastic cells, wherein said toxin-autoantibodies complex kill the pre-neoplastic or neoplastic tissue.

In one embodiment, a sample containing autoantibodies is taken from the subject and the autoantibodies are complexed with toxin. The toxin-autoantibody complex is then introduced into the general circulation of the subject, wherein said toxin-autoantibodies complex kill the pre-neoplastic or neoplastic tissue.

In one embodiment, a sample containing autoantibodies is taken from the subject and the autoantibodies are complexed with toxin. The toxin-autoantibody complex is then introduced into the blood vessels that perfuse the organ(s) containing the pre-neoplastic or neoplastic cells, wherein said toxin-autoantibodies complex kill the pre-neoplastic or neoplastic tissue.

In another embodiment, the autoantibodies are heterologous but are from the same species. In yet another embodiment, the autoantibodies are heterologous but are from different species. In yet another embodiment, the heterologous autoantibodies are pooled from multiple individuals.

In one embodiment, the toxin-autoantibody complex is introduced into the subject harboring the pre-neoplastic or neoplastic tissue or cells. In another embodiment, the toxin-autoantibody complex is introduced into the circulation of the subject. In another embodiment, the toxin-autoantibody complex is introduced into the blood vessels perfusing the organ with the pre-neoplastic or neoplastic tissue or cells.

In one embodiment, toxins are complexed with molecules that recognize auto-antibodies and the toxin-molecule complex are then administered to the mammal harboring the pre-neoplastic or neoplastic tissue. In one embodiment, the molecules are IgG (anti-IgG), Protein A, Protein G, Fab(2) fragment of an anti-IgG, Fab(1) of an anti-IgG, peptides that bind to the Fc region, and small molecules that bind antibodies. In one embodiment, the anti-IgG is an anti-human IgG or a humanized mouse anti-human IgG In one embodiment the anti-IgG includes the affinity ligand secretory component (Brandtzaeg, 1983), peptides that are designed based on hydropathy (Fassina et al., 1992), fragments of the placental alkaline phosphatase that can bind the Fc region (Makiya and Stigbrand, 1992), multimeric peptides (Verdoliva et al., 1995), one of the peptides from the E. coli surface exposed EiB proteins than bind IgG (Sandt and Hill, 2001), variations of the Fc receptor proteins that can bind IgG (Akilesh et al., 2007; Fridman, 1991; Fridman et al., 1984), anyone of a number of protein G or protein A mimetic (PAM) identified from peptide libraries that bind IgG (Fassina, 2000; Fassina et al., 1996; Fassina et al., 1998), or identified from combinatorial chemical synthesis (Fassina et al., 2001; Kabir, 2002), or combinatorial libraries (Nielsen et al., 2010), phage display libraries (Sakamoto et al., 2009), or rationally designed non-peptidyl mimetics of Protein A (Li et al., 1998), hexamer peptide affinity resins that bind the Fc region of IgG (Yang et al., 2005, 2009a) or the specific hexamer HWRGWV (Yang et al., 2010), use of the all-D amino acid peptide ligands (D'Agostino et al., 2008; Verdoliva et al., 2002), synthetic ligands including cyclic peptides (Verdoliva et al., 2005), a syntheic triazine scaffold substituted with 3-mainopheno and 4-amino-1-maphthol (Teng et al., 2000) or through other combinatorial chemical syntheses to make IgG binding ligands (Teng et al., 1999), affinity ligands that mimic Protein L (Roque et al., 2005), trisubstituted purine derivatives as protein A mimetics (Zacharie et al., 2010; Zacharie et al., 2009), or dendrimeric peptides (Moiani et al., 2009).

Complexing of the toxin to the autoantibody or molecule is performed by general methods of binding the autoantibody or molecules to a toxin. (Yoo et al., 2000)

In one embodiment, the toxin is paclitaxel, adriamycin, beta-emitters, or ricin.

In one embodiment, the toxin complexed with the autoantibody or molecule includes, but is not limited to, alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, camomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK™; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxanes, e.g. paclitaxel (TAXOL™, Bristol-Myers Squibb Oncology, Princeton, N.J.) and docetaxel (TAXOTERE™), Aventis, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In one embodiment the autoantibody, or the agent that recognizes the autoantibody is conjugated via a “slow-release” linkage to a toxin, such as Paclitaxel. The timing of the agent would be adjusted to maximize the extent to which antibody/agent—Paclitaxel which as not bound to the neoplasia has been cleared from the body and the binding of the antibody/agent—Paclitaxel to the neoplasma has been maximized prior to release of the toxin from the antibody (Grube et al., 2003; Jackson et al., 2000; O'Brien et al., 2003; Tanabe et al., 2003; Yang et al., 2009b).

Dosage can be by a single dose schedule or a multiple dose schedule. In a multiple dose schedule the various doses may be given by the same or different routes, e.g., intravenous, blood transfusion, or injection into the artery feeding the organ with the neoplasia. Multiple doses will typically be administered daily, every other day, three times a week, twice a week, or at least 1 week apart (e.g., about 2 weeks, about 3 weeks, about 4 weeks, about 6 weeks, about 8 weeks, about 10 weeks, about 12 weeks, about 16 weeks, etc.).

### 6. Pharmaceutical Compositions

In another aspect of the invention, the toxin-antibody or the toxin-molecule conjugates of the invention is a pharmaceutical composition suitable for administration to a mammal, preferably a human. To administer the toxin-antibody or the toxin-molecule conjugate composition to humans or animals, it is preferable to formulate the molecules in a composition comprising one or more pharmaceutically acceptable carriers. The phrase “pharmaceutically or pharmacologically acceptable” refers to molecular entities and compositions that do not produce allergic, or other adverse reactions when administered using routes well-known in the art. “Pharmaceutically acceptable carriers” include any and all clinically useful solvents, dispersion media, coatings, antibacterial and antifungal agents, isotonic and absorption delaying agents and the like.

Examples of pharmaceutically acceptable carriers or additives include water, a pharmaceutical acceptable organic solvent, collagen, polyvinyl alcohol, polyvinylpyrrolidone, a carboxyvinyl polymer, carboxymethylcellulose sodium, polyacrylic sodium, sodium alginate, water-soluble dextran, carboxymethyl starch sodium, pectin, methyl cellulose, ethyl cellulose, xanthan gum, gum Arabic, casein, gelatin, agar, diglycerin, glycerin, propylene glycol, polyethylene glycol, Vaseline, paraffin, stearyl alcohol, stearic acid, human serum albumin (HSA), mannitol, sorbitol, lactose, a pharmaceutically acceptable surfactant and the like. Additives used are chosen from, but not limited to, the above or combinations thereof, as appropriate, depending on the dosage form of the present invention.

Dosage can be by a single dose schedule or a multiple dose schedule. In a multiple dose schedule the various doses may be given by the same or different routes, e.g., intravenous, blood transfusion, or injection into the artery feeding the organ with the neoplasia. Multiple doses will typically be administered daily, every other day, three times a week, twice a week, or at least 1 week apart (e.g., about 2 weeks, about 3 weeks, about 4 weeks, about 6 weeks, about 8 weeks, about 10 weeks, about 12 weeks, about 16 weeks, etc.).

Efficacy of therapy can be assessed using any suitable method. Ways of checking efficacy of treatment involves monitoring levels of autoantibodies at the tissue or secretion level as discussed above.

Another way of assessing efficacy or therapy is to screen patient sera or secretions in a suitable immuno-assay (e.g., immunoblot, ELISA, microarray) for immunological reactivity to the antigen used to immunize the subject. A positive reaction between the antigen and the patient sample indicates that the patient has mounted an immune response to the antigen in question. This method may also be used to identify immunodominant antigens and/or epitopes within pathogens or antigens. Efficacy can also be determined in vivo using appropriate animal models of infection by the pathogen of interest, for example, by challenging the animal model with the pathogen of interest.

Efficacy of treatment can also be determine by the complete spectra of tools currently used to follow the efficacy of treatment including whole body scans (CAT Scan, x-ray, PET scans, MRI) and blood tests.

The compositions described herein can be administered in combination with one or more additional therapeutic agents. The additional therapeutic agents may include, but are not limited to antibiotics or antibacterial agents, antiemetic agents, antifungal agents, anti-inflammatory agents, antiviral agents, immunomodulatory agents, cytokines, antidepressants, hormones, alkylating agents, antimetabolites, antitumour antibiotics, antimitotic agents, topoisomerase inhibitors, cytostatic agents, anti-invasion agents, antiangiogenic agents, inhibitors of growth factor function inhibitors of viral replication, viral enzyme inhibitors, anticancer agents, α-interferons, β-interferons, ribavirin, hormones, and other toll-like receptor modulators, immunoglobulins (Igs), and antibodies modulating Ig function (such as anti-IgE (omalizumab)).

### 7. Kits

In one embodiment, the kit comprises a container with reagents for detecting an autoantibody and instructions for using and measuring the reagent and thus autoantibody to detect or diagnose a pre-neoplastic or neoplastic tissue.

In another embodiment, the kit comprises a container for enriching a subject's IgGs from its serum and then conjugating a label for detecting to the IgG. A subject's blood or serum is introduced into the container and the resulting labeled IgG is then reinjected back into the subject for detection. In one embodiment the subject is human. In another embodiment the subject is an animal.

Kits that utilize reagents for detection of the autoantibodies can be supplied for the present invention. The molecules that detect autoantibodies are typically provided in lyophilized form, either alone or in conjunction with buffers, stabilizers, inert proteins, or the like, in accordance with well-known manufacturing procedures. In one embodiment the agents that detect antibodies are conjugated to labels for detecting where the autoantibodies are bound in either histological sections, in fresh explants, during interoperative procedures or in whole subject scanning. In another embodiment the agents that detect antibodies are conjugated to toxins for killing the pre-neoplasia or neoplasia. In another embodiment the agents that detect autoantibodies are conjugated both to labels for detection and simultaneously conjugated to toxins.

Kits that utilize heterologous autoantibodies, from the same species but not from the subject, can be supplied for the present invention. The autoantibodies are typically provided in lyophilized form, either alone or in conjunction with buffers, stabilizers, inert proteins, or the like, in accordance with well-known manufacturing procedures. In one embodiment the heterologous autoantibodies are conjugated to labels for detection in either histological sections, in fresh explants, during interoperative procedures or in whole subject scanning. In another embodiment the heterologous autoantibodies are conjugated to toxins for killing the pre-neoplasia or neoplasia. In another embodiment the heterologous autoantibodies are conjugated both to labels for detection and simultaneously conjugated to toxins.

Kits that can utilize a subject's own autoantibodies can be supplied for the present invention. The kit comprises a container for enriching a subject's IgGs from its serum. In one embodiment the kit contains reagents for conjugated the subjects IgGs to labels for detection in either histological sections, in fresh explants, during interoperative procedures or in whole subject scanning. In another embodiment the kit contains reagents for conjugating toxins to the subjects IgGs for killing the pre-neoplasia or neoplasia. In another embodiment the kit contains reagents for conjugating both toxins and labels for detection to the subjects IgGs.

Kits can also be supplied for use with the autoantibodies of the present invention. Thus, the autoantibodies are typically provided in lyophilized form, either alone or in conjunction with buffers, stabilizers, inert proteins, or the like, in accordance with well-known manufacturing procedures. In a preferred method of the present invention, antibodies will be utilized in immunohistochemical staining procedures for use in detecting the markers in tissue samples. Tissue samples may be obtained from surgically-removed tissue, which has been frozen and sectioned. The fixed frozen section may be analyzed fixed, such as in formalin, acetone, or other standard histological preservatives, or analyzed unfixed as discussed above.

In one embodiment, the kit comprises a container containing the labeled autoantibody and instructions for using and measuring the autoantibody to detect or diagnose a pre-neoplastic or neoplastic tissue.

In another embodiment, the kit comprises a container with an autoantibody, another container contains the reagents for detecting the autoantibody, and instructions for using and measuring the autoantibody and reagent to detect or diagnose a pre-neoplastic or neoplastic tissue.

### EXAMPLES

The invention now being generally described, it will be more readily understood by reference to the following examples, which are included merely for purposes of illustration of certain aspects and embodiments of the present invention, and are not intended to limit the invention.

### Example 1

### Materials and Methods

1. Mouse Models:

The Alb-c-myc mouse models (Murakami et al., 1993) were a generous gift from Herman Stellar and both prostate models (Pb-_ENREF_{—}23MYC (Ellwood-Yen et al., 2003) in FBV background and conditional Pten knockout (Pb-Cre X Pten^{f/f}) in C57/B6 background (Trotman et al., 2003a, b)) were a generous gift from Charles Sawyers. Alb-myc and prostate sample controls were C57BL/6J mice purchased from The Jackson Laboratory. All FVB/N-Tg(MMTVneu)202Mul/J mice, and the corresponding control FVB/NJ mice were purchased from The Jackson Laboratory. In addition six week old BALB/c mice (Jackson Labs) and CBySmn.CB17-Prkdc^{scid}/J mice (Jackson Labs) were injected with a 4T1 cell line. One-hundred thousand cells were injected into a single mammary fat pad of each mouse, and mice were euthanized 14 days after injection. All experiments were approved by the Institutional Animal Care and Use Committee at The Rockefeller University.

2. Preparation of Tissue:

All tissue was dissected and placed in 4% paraformaldehyde for fixed tissue samples or Tissue Tek O.C.T. compound for frozen section samples. Frozen sections were then flash frozen in liquid nitrogen. A test sample and an age-matched corresponding WT sample were placed on the same slide for all experiments. In alb-myc and corresponding WT mice, at least one liver section was taken from each liver lobe.

3. Immunofluorescence:

The immunofluorescence detection of endogenous tissue was performed at the Molecular Cytology Core Facility of Memorial Sloan Kettering Cancer Center using Discovery XT processor (Ventana Medical Systems, Tucson Ariz.). Avidin Biotin block was then applied for 12 minutes. The tissue samples were then incubated with biotinylated secondary mouse (Vector Labs, MOM Kit BMK-2202) in 1:200 dilution (6.5 ug/mL). Detection was performed with Blocker D, Streptavidin-HRP D (Ventana Medical Systems) and followed by incubation with Tyramide-Alexa Fluor 488 (Invitrogen, cat #T20992). Negative controls were performed using biotinylated secondary rabbit IgG. (Vector Labs) IgM and IgA studies were processed as above (Invitrogen cat #M31515, M31115). Slides stained with CD34 were done in a similar fashion; however, a rat anti-mouse CD34 antibody (eBioscience, cat #14-0341) was used in 5 ug/ml concentrations. The protocol involves blocking (10% normal rabbit serum, 2% BSA) for 30 minutes, Protease 3 for 4 minutes, and a 7 hour incubation with primary antibody, followed by 16 minute incubation with biotinylated rabbit anti-rat IgG (Vector, cat #BA-4000, 1:200 dilution), Blocker D, Streptavidin-HRP (from DAB detection kit, Ventana Medical Systems), followed by incubation with Tyramide-Alexa Fluor 488 (Invitrogen, cat #T20922). Frozen section slides were dried at room temperature under the hood for 20 minutes, then baked at 56 degrees Celsius for an hour with the slide warmer, prior to immunofluorescence staining.

4. Image Analysis:

All slides were digitally scanned at the Molecular Cytology Core Facility of Memorial Sloan Kettering Cancer Center using the Zeiss Mirax Scanner with 20×/0.8NA objective and an exposure time of 5 ms with a gain of 2 for the transgenic mice and 10 ms for the xenograft mice.

5. Pathology Analysis:

Adjacent sections were given to a veterinary histopathologist in the Center of Comparative Medicine and Pathology at the Laboratory of Comparative Pathology who was blinded to all sources of tissue and all immunofluorescence results. These slides were stained with hematoxylin and eosin stain (“H&E”) and all tissue was analyzed and graded. All abnormal areas were marked and described by the degree of abnormality. In the liver histology sections, the following grading system, determined by the pathologist, was applied: 0=normal tissue, 1=areas found to have karyomegaly, cytomegaly, cystoplasmic vacuolization or other cellular changes, 2=a focus of defined cellular alteration, 3=an adenoma causing compression of adjacent parenchyma, and 4=carcinoma. The mammary tissue grading scale is as follows: 0=normal tissue, 1=hyperplastic regions without atypia/physiological hyperplasia, 2=hyperplasia with atypia, and 3=carcinoma. The prostate histology grading scale was: 0=normal tissue, grade 1=PIN, and grade 2=carcinoma.

6. EM:

Liver tissues from the alb-myc model and C57Bl/6 model of mice were fixed in 4.0% paraformaldehyde and 0.1% glutaraldehyde in 0.1 M sodium cacodylate buffer (pH 7.4) overnight. Sections were processed for immuno electron microscopy to recognize existing antibody as described previously (Uryu et al., 2001). Summation of tissue processing included quenching endogenous peroxidase with 0.5% hydrogen peroxide, blocking nonspecific antibody binding with 3% bovine serum albumin, applying biotinylated anti-mouse IgG, visualizing the immunocomplex with Vectastain ABC Kit (Vector Laboratories, Burlingame, Calif.) and peroxidate base reaction in the presence of 0.5% 3,3′ diaminobenzidine, and application of silver enhancement procedure to DAB immunoreactive products. Subsequently, sections were re-fixed with 2.5% glutaraldehyde in 0.1 M sodium cacodylate buffer, dehydrated by a graded series of ethanol, postfixed with 1% osmium tetra-oxide and embedded in EMBed812. Ultra-thin sections were cut and examined in the electron microscope (100CX JEOL, Tokyo, Japan) with the digital imaging system (XR41-C, Advantage Microscopy Technology Corp, Danver, Mass.). All EM was done in the Electron Microscopy Resource Center at The Rockefeller University.

7. Statistical Analysis:

All statistical analyses were performed using R version 2.12 using the library lme4 (www.r-project.org). All intensity values were normalized per pixel of tissue. Ratios of test sample to control sample were obtained for each slide comparing the number of pixels with intensity values of the top 40% of intensities. These were log-transformed to ensure that the distribution of ratios were approximately normal. One-way random effects model was used to generate p-values while taking into account the clustering resulting from the multiple observations contributed to the analysis by each mouse. (5 contributions in alb-myc tissue, 3 contributions in MMTV-neu, Pb-myc, and PTEN tissue). Random effects model parameters were estimated and tested by restricted maximum likelihood. All p values less than 0.05 were considered statistically significant. All ratios are presented as mean±SEM, with SEM calculated taking into account clustering, again, from the random effect models.

8. In Situ Imaging:

Balb/c mice bearing 4T1 xenograft tumors in the flank were euthanized and dissected to expose the tumor. Rat anti-mouse IgG conjugated to Cy5.5 was purchased from Jackson ImmunoResearch Laboratories (West Grove, Pa.), and goat anti-rabbit IgG was obtained from Invitrogen Corporation (Carlsbad, Calif.). Antibodies were diluted to a 0.67 nanomolar concentration in PBS, incubated with the tissue for 5 minutes, and washed with PBS. All images were acquired using the IVIS Lumina imaging system (Caliper Life Sciences, Inc).

### Example 2

### Alb-myc Model: Differentiating Abnormal Vs. Normal Tissue

MYC oncogene overexpression is frequently seen in patients with hepatocellular carcinoma (Shachaf et al., 2004). Therefore, the presence of autoantibodies in an alb-myc mouse model of cancer was probed.

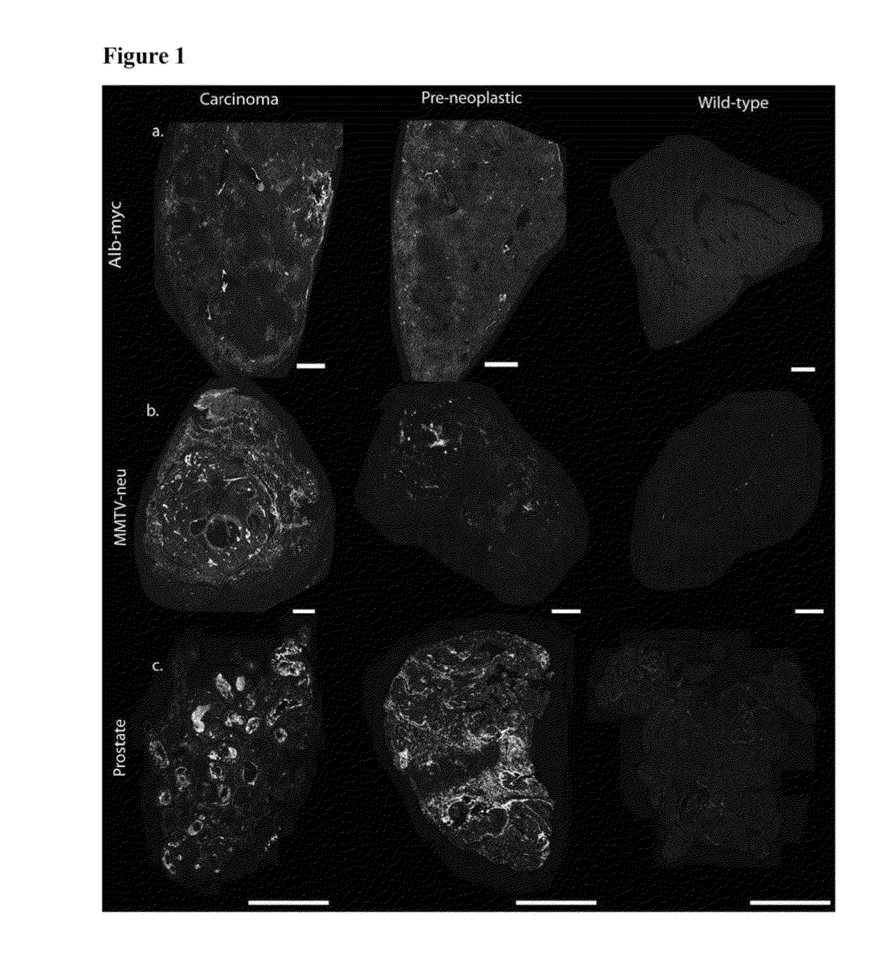

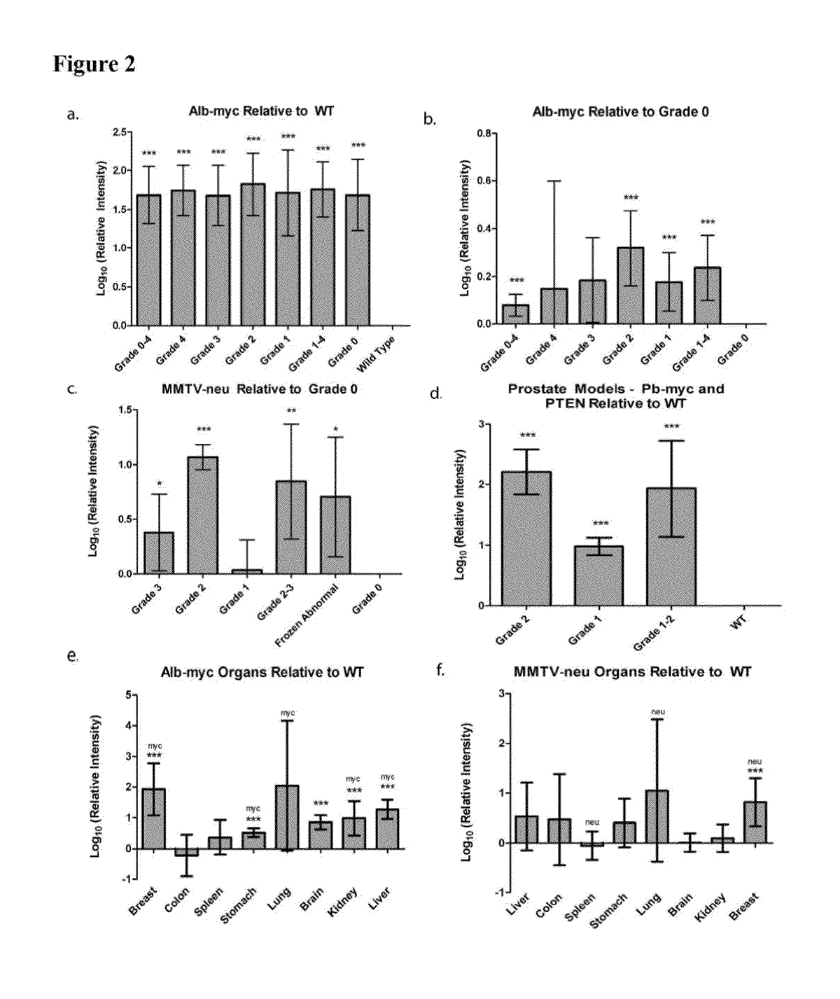

Liver tissue samples from this tumor model (Murakami et al., 1993) and from wild-type (“WT”) mice were paraffin embedded, mounted on the same slide, and probed with fluorescently tagged horse anti-mouse IgG antibody. In the alb-myc mice, anti-mouse IgG recognized autoantibodies throughout the tissue. Fluorescence was speckled with some areas considerably brighter than others. The anti-mouse IgG associated fluorescent intensity was approximately 50-fold higher in all liver tissue from alb-myc mice relative to tissue from WT mice (1.68±0.366 log 10-fold greater, p<0.001, 12 alb-myc/WT pairs, 3-5 tissue slices per mouse) (FIGS. 1a, 2a).

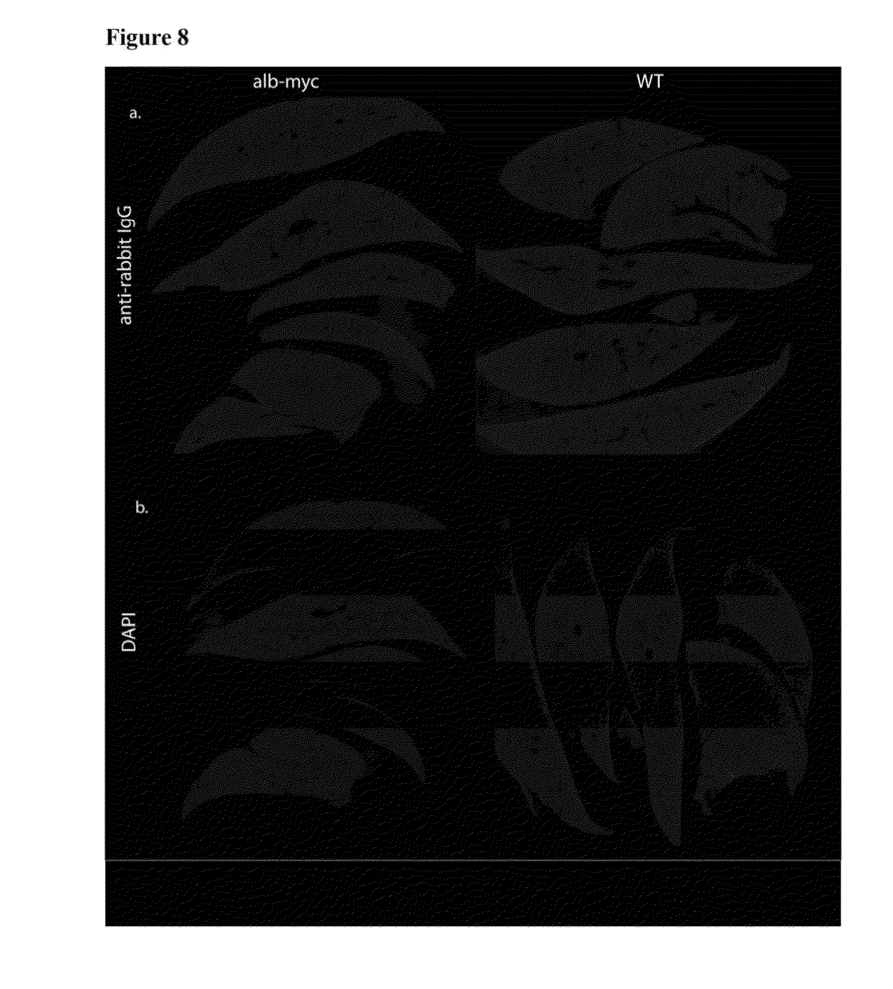

Similar results were observed with goat anti-mouse IgG. In contrast, there was no difference observed between alb-myc and WT tissue when probed with goat anti-rabbit IgG or chicken anti-rabbit IgG (Supplemental FIG. 1). This indicated specific recognition of mouse autoantibodies, rather than increased adhesion of the goal anti-IgG within tumor tissue. No detectable difference was observed between alb-myc and WT tissue in the absence of anti-mouse IgG, negating a contribution of autofluorescence (FIG. 8). Additionally, there was no difference between alb-myc and WT tissue when probed with anti-mouse IgM or anti-mouse IgA.

Adjacent sections were stained with H&E and analyzed by a histopathologist. All alb-myc tissue was graded as either 0 (normal), 1 (areas with cellular alterations), 2 (foci of alteration), 3 (adenoma causing compression) or 4 (carcinoma). All WT liver sections were histopathologically normal and will be referred to as WT. Two alb-myc mice had liver sections with grade 4 lesions, ten had grade 3 lesions, eight had grade 2 lesions, and five had grade 1 changes. Two alb-myc mice had no abnormalities on H&E. The fluorescent intensity of anti-mouse IgG was greater in all tumor grades relative to WT (grade 4: 1.74±0.322 log-fold greater, 3: 1.67±0.386, 2: 1.82±0.403, 1: 1.71±0.553; 0: 1.68±0.458; all p<0.001). When all abnormal grades were analyzed together (grades 1-4), the mean intensity was 1.76±0.351 log-fold greater than the WT group (p<0.001) (FIG. 2a).

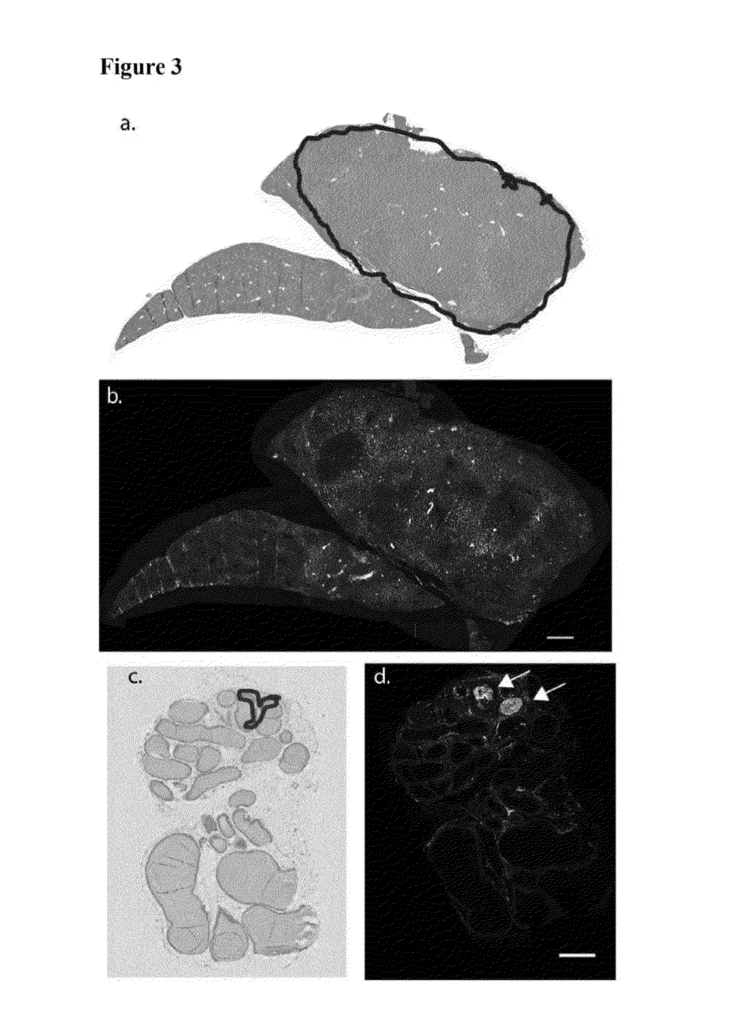

Regions graded 1-4 in the alb-myc tissue were also compared to grade 0 regions within the same tissue. Anti-mouse IgG localized to areas marked as abnormal on histopathology (FIGS. 3a, 3b). Areas graded 2 or 1 had a greater fluorescent intensity than areas graded 0 (0.317±0.157 log-fold greater and 0.176±0.121 log-fold greater respectively, p<0.001). Areas marked as grade 4 or 3 had a 0.148±0.45 log greater and 0.184±0.179 log-fold greater mean intensity respectively; however, this was not statistically significant. The fluorescent intensity of grades 1-4 was 0.235±0.136 log-fold greater (p<0.001) than grade 0 (FIG. 2b). Grade 0 tissue, however, had 1.68±0.458 log-fold greater intensity than matched WT tissue (FIG. 2a). Consequently, grade 0 regions in three alb-myc mice were probed and found to be reactive with anti-CD34 antibody, a marker of early transitioned sinusoidal endothelial cells (Frachon et al., 2001) not seen on normal liver sinusoidal endothelial cells. WT liver sinusoidal endothelial cells did not react with this antibody. Thus, even though these regions of the alb-myc mice were rated grade 0, and therefore show no obvious alterations on H&E, they are likely to be at early stages of transformation as diagnosed both by the autoantibodies within the tissue and the reactivity with the anti-CD34 antibody.

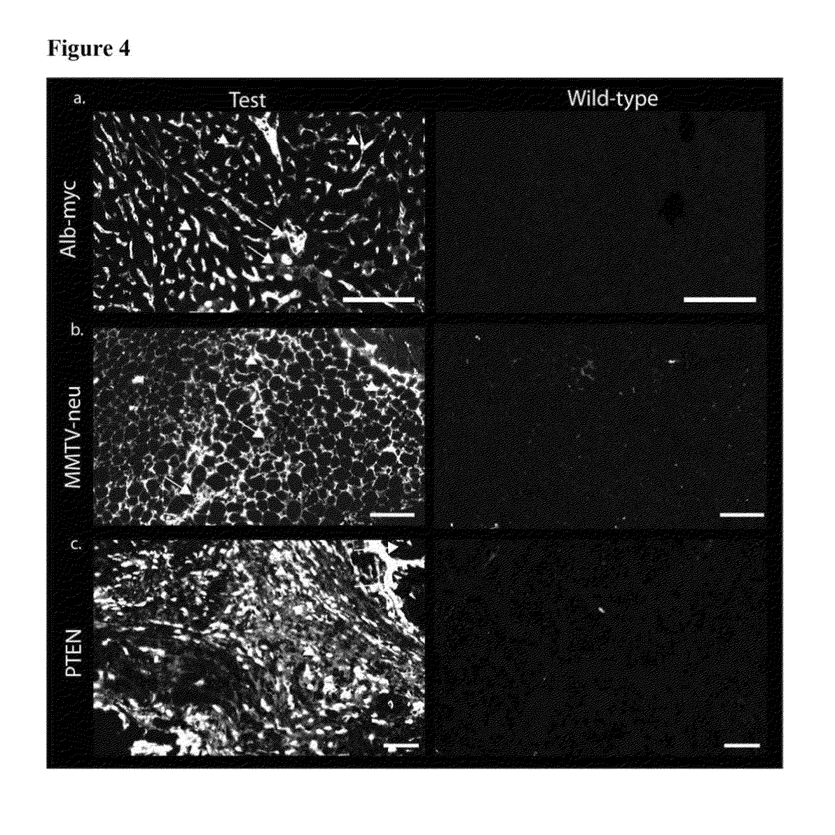

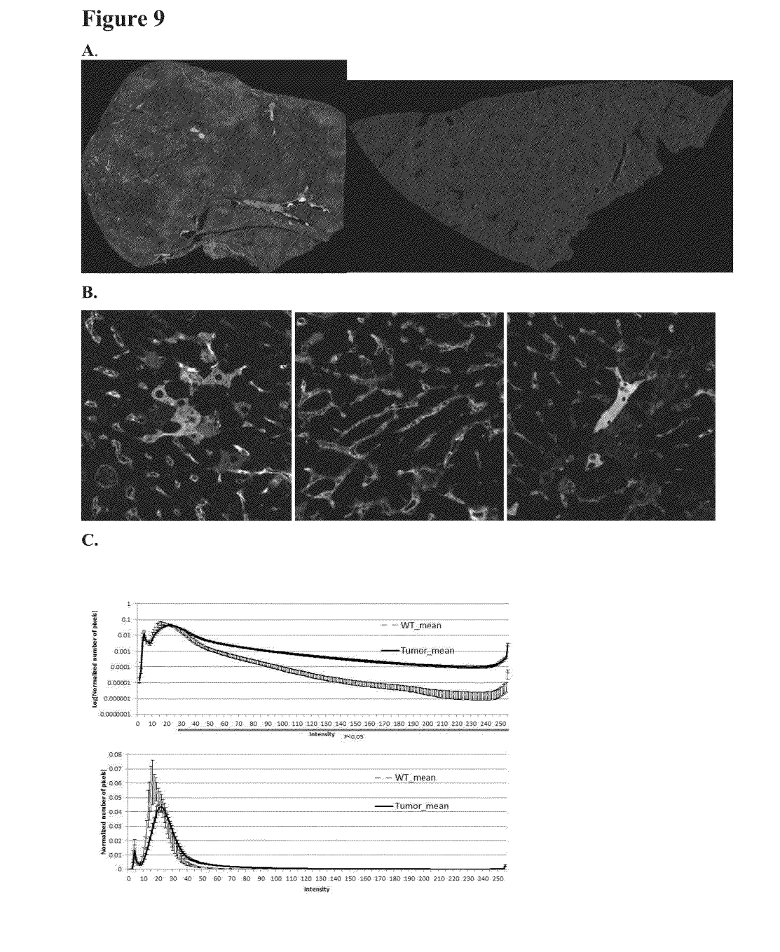

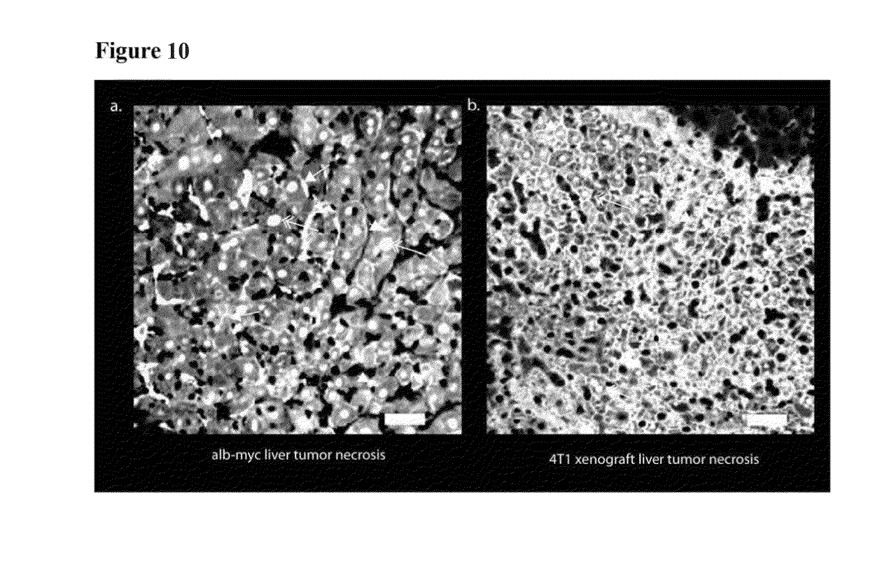

The variability of binding observed under the lower magnification (FIG. 3b) was also seen under higher magnification (FIG. 4a, FIG. 9). In some regions, the fluorescence was associated with sinusoidal endothelial cells, in other regions with hepatocytes, and in some regions, both cell types were brightly fluorescent. The binding of anti-mouse IgG to sinusoidal endothelial cells was confirmed with immuno-electron microscopy (EM) (FIG. 9). Occasionally, small tumor regions were found to be necrotic. In these areas there were autoantibodies bound to the hepatocyte membrane, cytoplasm, and nucleus (FIG. 10).

### Example 3

### MMTV-neu Model: Differentiating Abnormal Vs. Normal Tissue

Human breast tumors contain amplification of HER-2/neu in 25-30% of patients (Slamon et al., 1989). Thus, the presence of endogenous antibodies in mammary tissue from virgin and multiparous mice expressing the un-activated neu oncogene, driven by a mouse mammary tumor virus (MMTV) promoter was probed (Guy et al., 1992).

Breast tissue was paraffin embedded and probed with fluorescently labeled anti-mouse IgG; adjacent sections were stained with H&E (n=10 MMTV-neu/WT pairs, 1-2 mammary glands taken from each mouse). Regions were histopathologically graded as either 0 (normal), 1 (hyperplasia without atypia/physiological hyperplasia), 2 (hyperplasia with atypia), or 3 (carcinoma). In the eighteen MMTV-neu tissue samples, six had grade 3 lesions, four had grade 2 lesions, and the remaining samples were grade 0. In the eighteen WT samples, five had grade 2 lesions, eleven had grade 1 regions, and two samples were uniformly grade 0. In the alb-myc model where all cells expressed the activated oncogene, there was a significant difference between grade 0 in the tumor model and grade 0 in the wild-type. In this model where unactivated neu was expressed there were no obvious differences between grade 0 in the wt and tumor model. Further, in many of the WT mice many abnormalities were observed in the breast. Therefore, on each slide, all regions that were histopathologically given the same grade were analyzed as a group, whether the tissue was from a MMTV-neu mouse or a WT mouse.

Grade 3 and 2 lesions had greater antibody binding than histopathologically normal, grade 0 mammary tissue (FIG. 1b). The grade 3 samples had 0.378±0.0351 log-fold greater intensity than corresponding normal mammary tissue (p=0.02). Tissue with grade 2 lesions had 1.07±0.114 log-fold greater intensity than normal tissue (p<0.001) (FIG. 2c). There was no observed difference in fluorescent intensity between grades 1 and 0; however, grade 1 includes physiologic hyperplasia, which is not a pathological state. When comparing grade 2 and 3 (abnormal tissue) with grade 0, there was 0.84 log-fold greater intensity (p<0.001). Similar to the alb-myc model, there was no detectable difference in autoantibody binding between MMTV-neu and WT tissue probed with anti-rabbit IgG, or processed without antibody.

Mouse IgG was present in abnormal mammary tissue bound to abnormal ductal and alveolar cells (arrows in FIG. 4b), the surrounding adipocytes (arrowheads in figure), collagen, and skeletal muscle, as well as within alveolar and ductal glands. Antibody binding to tumor cells was variable, with fewer bound in the center of large tumors (FIG. 1b). Histopathologically normal lymph nodes within the mammary tissue did not contain antibody binding.

### Example 4

### Mouse Models for Prostate Cancer: Differentiating Abnormal Vs. Normal Tissue

The presence of tissue-bound autoantibodies in prostate cancer using two different mouse models was tested.

The first model expressed human myc under the prostate-specific Pb promoter (Ellwood-Yen et al., 2003). Myc is overexpressed in 30% of human prostate cancers and expression in mice leads to prostatic intraepithelial neoplasia (PIN), followed by invasive adenocarcinoma (Ellwood-Yen et al., 2003).

The second prostate tumor model was a knock-out of the PTEN tumor suppressor. This gene is deleted in 70-80% of human prostate cancers (Gray et al., 1998; Whang et al., 1998) and prostate specific deletion also results in murine prostate cancer (Trotman et al., 2003a). The previous mouse tumor models used were based on overexpression of an oncogene. In this example, a deleted gene was explored to evaluate whether tissue autoantibodies were identifying neoplasia as an inflammatory response to overexpression of a transgene.

In six prostate model samples (four Pb-myc and two PTEN knockout), two Pb-myc were grade 2 (tumor) and the remaining four were grade 1 (PIN). All WT prostates were histopathologically normal. Grade 2 lesions had 2.21±0.373 log-fold greater intensity (p<0.001) than the respective WT (FIGS. 1c, 2d). Grade 1 PIN samples had 0.979±0.147 log-fold greater intensity (p<0.001) than WT. All abnormal tissue (grade 1-2) had 1.935±0.796 log-fold greater intensity (p<0.001) than the matched WT (FIG. 2d). Again, there was no difference in fluorescence when tissue was processed without any antibody.

The levels of antibody binding varied throughout the tumor and PIN cells (FIG. 1c, 4c arrows). There was considerable binding of autoantibodies throughout the tumor microenvironment, including the stroma and fibrovascular structures surrounding abnormal regions (FIG. 4c, arrowheads), and the prostatic secretions adjacent to altered regions (FIGS. 3c, 3d, arrows).

### Example 5

### Probing of Organs in Transgenic Models

To test if the presence of a tumor causes an immune response that increases autoantibody binding elsewhere in the body, the presence of autoantibodies was probed in other organs, including the brain, stomach, colon, spleen, kidney, lung, liver, and mammary tissue. All organs, other than MMTV-neu mammary tissue and alb-myc liver tissue, were found to be histopathologically normal.

There was no detectable difference in autoantibody binding in the liver, colon, spleen, stomach, lung, brain, and kidney in the MMTV-neu mice compared to WT. The only tissue with greater antibody binding than normal tissue was abnormal (grade 2-3) mammary gland, which had 0.819±0.485 log-fold greater intensity than grade 0 (normal) tissue (FIG. 2e). The MMTV promoter is predominantly expressed in mammary tissue. It has also been reported to be partially expressed in lung, salivary gland, and spleen but at a 500-fold lower expression level (Henrard and Ross, 1988).

In the alb-myc mice, there was a greater fluorescence in mammary tissue (1.93±0.85 log-fold), brain (0.859±0.23 log-fold) kidney (0.987±0.56 log-fold) and liver (1.28±0.31 log-fold), relative to WT (all p<0.001) (FIG. 2f). The alb-myc mouse is a model for hepatocellular carcinoma due to the high level of albumin expression in the liver. However, it has been reported to be expressed in the mammary gland, heart, lungs, gastrointestinal tract, kidney, brain and pancreas (Nahon et al., 1988; Poliard et al., 1988; Shamay et al., 2005). This expression pattern correlates with the observed autoantibody binding.

### Example 6

### Xenograft Model

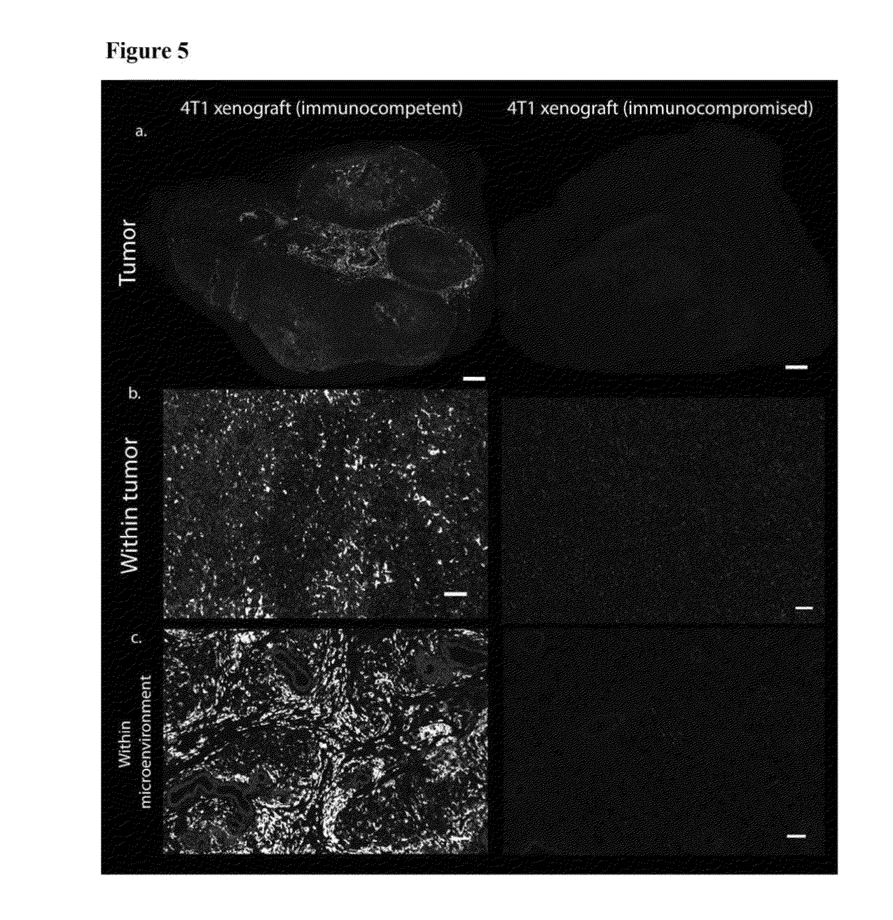

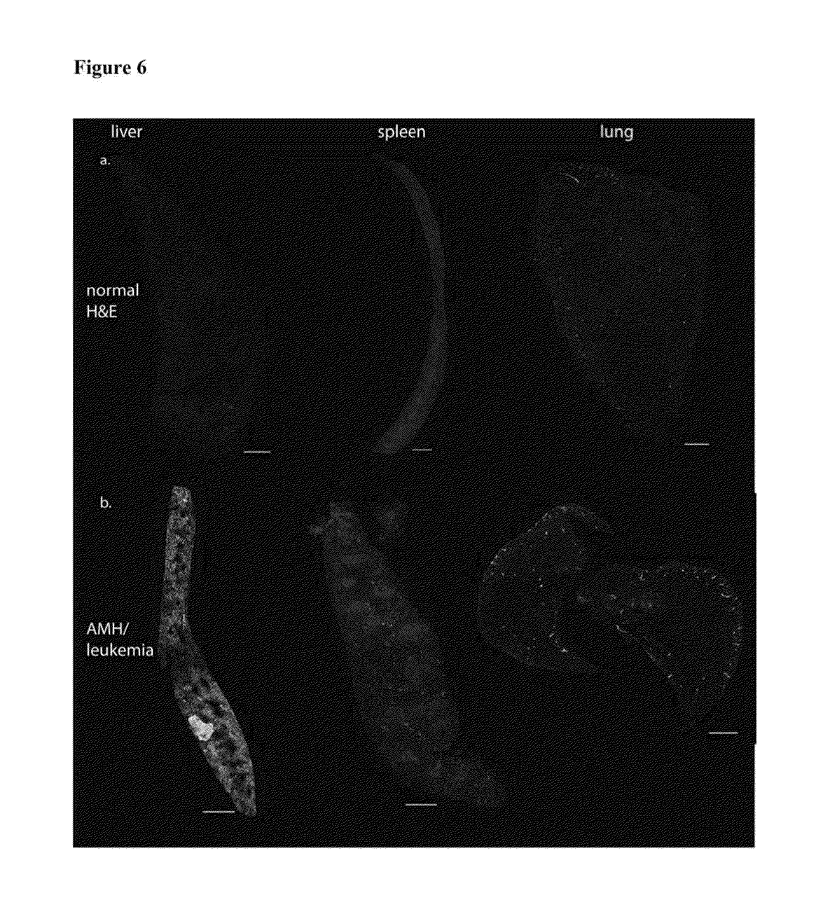

A xenograft mouse model of cancer was also tested, which gave greater control of tumor cell localization than in transgenic models. The mouse breast cancer 4T1 cell line was injected into a single mammary fat pad in 3 immunocompetent and 3 SCID mice, and mice were dissected on day 14. Assays for autoantibody binding demonstrated mouse IgG bound within the tumor and the microenvironment in immunocompetent mice; however, no detectable antibody was bound in immunosuppressed mice (FIG. 5). In two immunocompetent mice, all organs (including contralateral mammary tissue) were histopathologically normal and demonstrated limited antibody binding (FIG. 6). In the third immunocompetent mouse, the liver, spleen, and lung had diffuse regions of increased antibody binding (FIG. 6). Adjacent H&E sections of these three organs were determined to have atypical myeloid hyperplasia (AMH)/myeloid leukemia. The liver section also contained an area of necrosis secondary to tumor infiltration (FIG. 10). The other organs tested in this mouse (contralateral mammary tissue, skin, kidney, stomach, and colon) did not have any areas of distinct antibody binding and were noted to be normal by histopathology. Similar results were seen in a B16 xenograft model of melanoma (data not shown).

### Example 7

### Tumor Imaging In Situ and Frozen Sections

The utility of autoantibodies as a diagnostic tool in frozen sections was tested in seven pairs of abnormal and normal mammary tissue (FIG. 7 a,b). The intensity of fluorescent labeling in abnormal tissue (FIG. 7a) was 0.703 log-fold greater than normal tissue (FIG. 7b) on the same slide (p=0.02) (FIG. 2c).

Mice bearing 4T1 xenografts were used to determine whether the increased antibody binding identified in tissue slices could be used to localize tumor in situ. IgG antibodies from the mice were enriched and labeled with one fluorophore and reinjected back into the mice (FIG. 11) along with a non-specific anti-chicken Igγ labeled with a different fluorophore. In whole animal imaging the mouse antibodies selectively labeled the tumor demonstrating the utility of localizing the tumor in situ. Similar results were observed with PET imaging.

Mice bearing 4T1 xenografts were used to determine if injection of the labeled autoantibodies could be used for intraoperative image guided surgery. Upon opening the abdomen, a fluorescence signal was selectively observed only for the autoantibodies, and not for the non-specific antibodies, and only at the site of the tumor (FIG. 12).

Mice bearing 4T1 xenografts were used to determine if agents that can detect autoantibodies can be used intraoperative to guide surgery. The tumor was partially resected from the mouse's flank, and the entire flank region was incubated with a rat anti-mouse IgG conjugated to Cy5.5. To account for non-specific staining of the tissue by the conjugated antibody, a goat anti-rabbit IgG conjugated to AlexaFluor 568 was incubated simultaneously in the same region. Subtracting the anti-rabbit signal from the anti-mouse signal gave specific localization of the remnant xenograft tumors (FIG. 7c).

### Example 8

### Tumor Imaging in Human Cancers

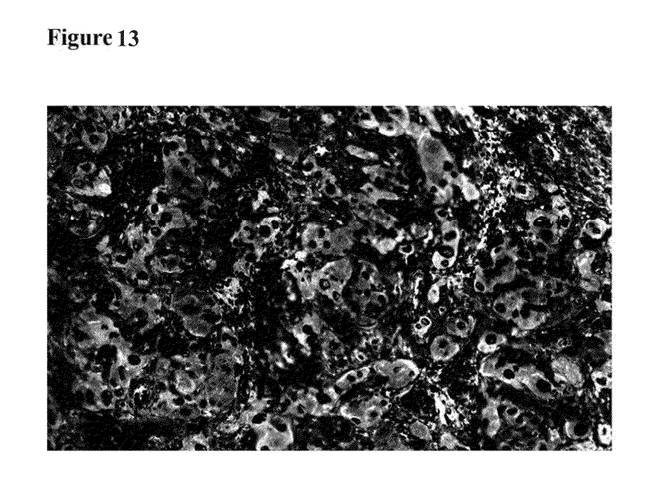

The presence of human auto-antibodies bound in a neoplasia was tested in a liver cancer. A biopsy of a fibrolamellar hepatocellular carcinoma from a human was probed with a fluorescently tagged anti-human IgG (FIG. 13). All of the transformed tissue had bound human antibodies.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and GenBank sequences cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Those skilled in the art will recognize, or be able to ascertain using no more than routine experimentation, many equivalents to the specific embodiments of the invention described herein. Such equivalents are intended to be encompassed by the following embodiments.

### REFERENCES

1. Loeb, L. A., Loeb, K. R. & Anderson, J. P. Multiple mutations and cancer. Proc Natl Acad Sci USA 100, 776-781 (2003).
2. Ehrlich, P. About the current state of carcinoma research. in Ned Tijdscher Geneeskd, Vol. 5 (1909).
3. Birkeland, S. A., et al. Cancer risk after renal transplantation in the Nordic countries, 1964-1986. Intl Cancer 60, 183-189 (1995).
4. Penn, I. Malignant melanoma in organ allograft recipients. Transplantation 61, 274-278 (1996).
5. Penn, I. Sarcomas in organ allograft recipients. Transplantation 60, 1485-1491 (1995).
6. Pham, S. M., et al. Solid tumors after heart transplantation: lethality of lung cancer. Ann Thorac Surg 60, 1623-1626 (1995).
7. Dunn, G. P., Bruce, A. T., Ikeda, H., Old, L. J. & Schreiber, R. D. Cancer immunoediting: from immunosurveillance to tumor escape. Nat Immunol 3, 991-998 (2002).
8. Akcakanat, A., et al. NY-ESO-1 expression and its serum immunoreactivity in esophageal cancer. Cancer Chemother Pharmacol 54, 95-100 (2004).
9. Stockert, E., et al. A survey of the humoral immune response of cancer patients to a panel of human tumor antigens. J Exp Med 187, 1349-1354 (1998).
10. Chapman, C. J., et al. Autoantibodies in lung cancer: possibilities for early detection and subsequent cure. Thorax 63, 228-233 (2008).
11. Türeci, O., et al. Humoral immune responses of lung cancer patients against tumor antigen NY-ESO-1. Cancer Lett 236, 64-71 (2006).
12. Korangy, F., et al. Spontaneous tumor-specific humoral and cellular immune responses to NY-ESO-1 in hepatocellular carcinoma. Clin Cancer Res 10, 4332-4341 (2004).
13. Nakamura, S., et al. Expression and immunogenicity of NY-ESO-1 in hepatocellular carcinoma. J Gastroenterol Hepatol 21, 1281-1285 (2006).
14. Maio, M., et al. Analysis of cancer/testis antigens in sporadic medullary thyroid carcinoma: expression and humoral response to NY-ESO-1. J Clin Endocrinol Metab 88, 748-754 (2003).
15. Fosså, A., et al. NY-ESO-1 protein expression and humoral immune responses in prostate cancer. Prostate 59, 440-447 (2004).
16. Kobold, S., Lütkens, T., Cao, Y., Bokemeyer, C. & Atanackovic, D. Autoantibodies against tumor-related antigens: incidence and biologic significance. Hum Immunol 71, 643-651 (2010).
17. Zhang, J. Y. & Tan, E. M. Autoantibodies to tumor-associated antigens as diagnostic biomarkers in hepatocellular carcinoma and other solid tumors. Expert Rev Mol Diagn 10, 321-328 (2010).
18. Shachaf, C. M., et al. MYC inactivation uncovers pluripotent differentiation and tumour dormancy in hepatocellular cancer. Nature 431, 1112-1117 (2004).
19. Murakami, H., et al. Transgenic mouse model for synergistic effects of nuclear oncogenes and growth factors in tumorigenesis: interaction of c-myc and transforming growth factor alpha in hepatic oncogenesis. Cancer Res 53, 1719-1723 (1993).
20. Frachon, S., et al. Endothelial cell marker expression in dysplastic lesions of the liver: an immunohistochemical study. J Hepatol 34, 850-857 (2001).
21. Slamon, D. J., et al. Studies of the HER-2/neu proto-oncogene in human breast and ovarian cancer. Science 244, 707-712 (1989).
22. Guy, C. T., et al. Expression of the neu protooncogene in the mammary epithelium of transgenic mice induces metastatic disease. Proc Natl Acad Sci USA 89, 10578-10582 (1992).
23. Ellwood-Yen, K., et al. Myc-driven murine prostate cancer shares molecular features with human prostate tumors. Cancer Cell 4, 223-238 (2003).
24. Gray, I. C., et al. Mutation and expression analysis of the putative prostate tumour-suppressor gene PTEN. Br J Cancer 78, 1296-1300 (1998).
25. Whang, Y. E., et al. Inactivation of the tumor suppressor PTEN/MMAC1 in advanced human prostate cancer through loss of expression. Proc Natl Acad Sci USA 95, 5246-5250 (1998).
26. Trotman, L. C., et al. Pten dose dictates cancer progression in the prostate. PLoS Biol 1, E59 (2003).
27. Henrard, D. & Ross, S. R. Endogenous mouse mammary tumor virus is expressed in several organs in addition to the lactating mammary gland. J Virol 62, 3046-3049 (1988).
28. Shamay, A., et al. Expression of albumin in nonhepatic tissues and its synthesis by the bovine mammary gland. J Dairy Sci 88, 569-576 (2005).
29. Nahon, J. L., et al. Albumin and alpha-fetoprotein gene expression in various nonhepatic rat tissues. J Biol Chem 263, 11436-11442 (1988).
30. Poliard, A., Feldmann, G. & Bernuau, D. Alpha fetoprotein and albumin gene transcripts are detected in distinct cell populations of the brain and kidney of the developing rat. Differentiation 39, 59-65 (1988).
31. Scoazec, J. Y. & Feldmann, G. In situ immunophenotyping study of endothelial cells of the human hepatic sinusoid: results and functional implications. Hepatology 14, 789-797 (1991).
32. Bissell, M. J. & Radisky, D. Putting tumours in context. Nat Rev Cancer 1, 46-54 (2001).
33. Mueller, M. M. & Fusenig, N. E. Friends or foes—bipolar effects of the tumour stroma in cancer. Nat Rev Cancer 4, 839-849 (2004).
34. Roskelley, C. D. & Bissell, M. J. The dominance of the microenvironment in breast and ovarian cancer. Semin Cancer Biol 12, 97-104 (2002).
35. Coussens, L. M. & Werb, Z. Inflammation and cancer. Nature 420, 860-867 (2002).
36. Franses, J. W., Baker, A. B., Chitalia, V. C. & Edelman, E. R. Stromal endothelial cells directly influence cancer progression. Sci Transl Med 3, 66ra65 (2011).
37. Fiegl, H., et al. Breast cancer DNA methylation profiles in cancer cells and tumor stroma: association with HER-2/neu status in primary breast cancer. Cancer Res 66, 29-33 (2006).
38. Hu, M., et al. Distinct epigenetic changes in the stromal cells of breast cancers. Nat Genet. 37, 899-905 (2005).
39. Trimboli, A. J., et al. Pten in stromal fibroblasts suppresses mammary epithelial tumours. Nature 461, 1084-1091 (2009).
40. Orimo, A., et al. Stromal fibroblasts present in invasive human breast carcinomas promote tumor growth and angiogenesis through elevated SDF-1/CXCL12 secretion. Cell 121, 335-348 (2005).
41. Tyan, S. W., et al. Breast cancer cells induce cancer-associated fibroblasts to secrete hepatocyte growth factor to enhance breast tumorigenesis. PLoS One 6, e15313 (2011).
42. Dakhova, O., et al. Global gene expression analysis of reactive stroma in prostate cancer. Clin Cancer Res 15, 3979-3989 (2009).
43. Chung, L. W., Baseman, A., Assikis, V. & Zhau, H. E. Molecular insights into prostate cancer progression: the missing link of tumor microenvironment. J Urol 173, 10-20 (2005).
44. Jain, R. K. Physiological barriers to delivery of monoclonal antibodies and other macromolecules in tumors. Cancer Res 50, 814s-819s (1990).
45. Fujimori, K., Covell, D. G., Fletcher, J. E. & Weinstein, J. N. A modeling analysis of monoclonal antibody percolation through tumors: a binding-site barrier. J Nucl Med 31, 1191-1198 (1990).
46. Weinstein, J. N. & van Osdol, W. The macroscopic and microscopic pharmacology of monoclonal antibodies. Int J Immunopharmacol 14, 457-463 (1992).
47. Reuschenbach, M., von Knebel Doeberitz, M. & Wentzensen, N. A systematic review of humoral immune responses against tumor antigens. Cancer Immunol Immunother 58, 1535-1544 (2009).
48. Tsuboi, T., et al. Is intraoperative frozen section analysis an efficient way to reduce positive surgical margins? Urology 66, 1287-1291 (2005).
49. Liu, Y., et al. Hands-free, wireless goggles for near-infrared fluorescence and real-time image-guided surgery. Surgery 149, 689-698 (2011).
50. Trotman, L. C., et al. Pten dose dictates cancer progression in the prostate. PLoS Biol 1, E59 (2003).
51. Uryu, K., MacKenzie, L. & Chesselet, M. F. Ultrastructural evidence for differential axonal sprouting in the striatum after thermocoagulatory and aspiration lesions of the cerebral cortex in adult rats. Neuroscience 105, 307-316 (2001).
52. Achiefu, S. Hands-free, wireless goggles for near-infrared fluorescence and real-time image-guided surgery. Surgery 2011; 149:689-98.
53. Ye, Y., Xu, B. Exploring new near-infrared fluorescent disulfide-based cyclic RGD peptide analogs for potential integrin-targeted optical imaging. Bioorganic & Medicinal Chemistry Letters 21 (2011) 2116-2120.
54. Wang, Y. Large Scale Identification of Human Hepatocellular Carcinoma-Associated Antigens by Autoantibodies. The Journal of Immunology, 2002, 169: 1102-1109.
55. Kobold, S. Prognostic and Diagnostic Value of Spontaneous Tumor-Related Antibodies. Clinical and Developmental Immunology, Volume 2010, Article ID 721531, 8 pgs, doi: 10.1155/2010/721531.
56. Tan, E. Autoantibodies as reporters identifying aberrant cellular mechanisms in tumorigenesis. J. Clin Invest. 108:1411-1415 (2001). D01:10.1172/JC1200114451.
57. Zhang, J. Autoantibodies to tumor-associated antigens as diagnostic biomarkers in hepatocellular carcinoma and other solid tumors. Expert Rev Mol Diagn. 2010 April; 10(3): 321-328. doi: 10.1586/erm.10.12.
58. Grube, E., et al., (2003). TAXUS I: six- and twelve-month results from a randomized, double-blind trial on a slow-release paclitaxel-eluting stent for de novo coronary lesions. Circulation 107, 38-42.
59. Jackson, J. K., et al., (2000). The suppression of human prostate tumor growth in mice by the intratumoral injection of a slow-release polymeric paste formulation of paclitaxel. Cancer Res 60, 4146-4151.
60. O'Brien, M. E., et al. (2003). Carboplatin and paclitaxol (Taxol) as an induction regimen for patients with biopsy-proven stage IIIA N2 non-small cell lung cancer. an EORTC phase II study (EORTC 08958). Eur J Cancer 39, 1416-1422.
61. Tanabe, K., et al. (2003). TAXUS III Trial: in-stent restenosis treated with stent-based delivery of paclitaxel incorporated in a slow-release polymer formulation. Circulation 107, 559-564.
62. Yang, H., Li, K., Liu, Y., Liu, Z., and Miyoshi, H. (2009). Poly(D,L-lactide-co-glycolide) nanoparticles encapsulated fluorescent isothiocyanate and paclitaxol: preparation, release kinetics and anticancer effect. J Nanosci Nanotechnol 9, 282-287.
63. D'Agostino, B., Bellofiore, P., De Martino, T., Punzo, C., Rivieccio, V., and Verdoliva, A. (2008). Affinity purification of IgG monoclonal antibodies using the D-PAM synthetic ligand: chromatographic comparison with protein A and thermodynamic investigation of the D-PAM/IgG interaction. J Immunol Methods 333, 126-138.
64. Fassina, G. (2000) Protein A mimetic (PAM) affinity chromatography. Immunoglobulins purification. Methods Mol Biol 147, 57-68.
65. Fassina, G., Consonni, R., Zetta, L., and Cassani, G. (1992). Design of hydropathically complementary peptides for Big Endothelin affinity purification. Int J Pept Protein Res 39, 540-548.
66. Fassina, G., Ruvo, M., Palombo, G., Verdoliva, A., and Marino, M. (2001). Novel ligands for the affinity-chromatographic purification of antibodies. J Biochem Biophys Methods 49, 481-490.
67. Fassina, G., Verdoliva, A., Odierna, M. R., Ruvo, M., and Cassini, G. (1996). Protein A mimetic peptide ligand for affinity purification of antibodies. J Mol Recognit 9, 564-569.
68. Fassina, G., Verdoliva, A., Palombo, G., Ruvo, M., and Cassani, G. (1998). Immunoglobulin specificity of TG19318: a novel synthetic ligand for antibody affinity purification. J Mol Recognit 11, 128-133.
69. Fridman, W. H. (1991). Fc receptors and immunoglobulin binding factors. FASEB J 5, 2684-2690.
70. Fridman, W. H., Mathiot, C., Montcuit, J., and Teillaud, J. L. (1988). Fc receptors, immunoglobulin-binding factors and B chronic lymphocytic leukemia. Nouv Rev Fr Hematol 30, 311-315.
71. Gimble, J. M., Flanagan, J. R., Recker, D., and Max, E. E. (1988). Identification and partial purification of a protein binding to the human immunoglobulin kappa enhancer kappa E2 site. Nucleic Acids Res 16, 4967-4988
72. Kirschner, C., Maquelin, K., Pina, P., Ngo Thi, N. A., Choo-Smith, L. P., Sockalingum, G. D., Sandt, C., Ami, D., Orsini, F., Doglia, S. M., et al. (2001). Classification and identification of enterococci: a comparative phenotypic, genotypic, and vibrational spectroscopic study. J Clin Microbiol 39, 1763-1770.
73. Li, R., Dowd, V., Stewart, D. J., Burton, S. J., and Lowe, C. R. (1998). Design, synthesis, and application of a protein A mimetic. Nat Biotechnol 16, 190-195.
74. Moiani, D., Salvalaglio, M., Cavallotti, C., Bujacz, A., Redzynia, I., Bujacz, G., Dinon, F., Pengo, P., and Fassina, G. (2009). Structural characterization of a Protein A mimetic peptide dendrimer bound to human IgG. J Phys Chem B 113, 16268-16275.
75. Palombo, G., De Falco, S., Tortora, M., Cassani, G., and Fassina, G. (1998a). A synthetic ligand for IgA affinity purification. J Mol Recognit 11, 243-246.
76. Palombo, G., Rossi, M., Cassani, G., and Fassina, G. (1998b). Affinity purification of mouse monoclonal IgE using a protein A mimetic ligand (TG19318) immobilized on solid supports. J Mol Recognit 11, 247-249.
77. Palombo, G., Verdoliva, A., and Fassina, G. (1998c). Affinity purification of immunoglobulin M using a novel synthetic ligand. J Chromatogr B Biomed Sci Appl 715, 137-145.
78. Roque, A. C., Taipa, M. A., and Lowe, C. R. (2005). Synthesis and screening of a rationally designed combinatorial library of affinity ligands mimicking protein L from Peptostreptococcus magnus. J Mol Recognit 18, 213-224.
79. Rossi, M., Ruvo, M., Marasco, D., Colombo, M., Cassani, G., and Verdoliva, A. (2008). Anti-allergic properties of a new all-D synthetic immunoglobulin-binding peptide. Mol Immunol 45, 226-234.
80. Sakamoto, K., Ito, Y., Hatanaka, T., Soni, P. B., Mori, T., and Sugimura, K. (2009). Discovery and characterization of a peptide motif that specifically recognizes a non-native conformation of human IgG induced by acidic pH conditions. J Biol Chem 284, 9986-9993.
81. Sandt, C. H., and Hill, C. W. (2001). Nonimmune binding of human immunoglobulin A (IgA) and IgG Fc by distinct sequence segments of the EibF cell surface protein of Escherichia coli. Infect Immun 69, 7293-7303.
82. Teng, S. F., Sproule, K., Hussain, A., and Lowe, C. R. (1999). A strategy for the generation of biomimetic ligands for affinity chromatography. Combinatorial synthesis and biological evaluation of an IgG binding ligand. J Mol Recognit 12, 67-75.
83. Verdoliva, A., Cassani, G., and Fassina, G. (1995). Affinity purification of polyclonal antibodies using immobilized multimeric peptides. J Chromatogr B Biomed Appl 664, 175-183.
84. Verdoliva, A., Marasco, D., De Capua, A., Saporito, A., Bellofiore, P., Manfredi, V., Fattorusso, R., Pedone, C., and Ruvo, M. (2005). A new ligand for immunoglobulin g subdomains by screening of a synthetic peptide library. Chembiochem 6, 1242-1253.
85. Verdoliva, A., Pannone, F., Rossi, M., Catello, S., and Manfredi, V. (2002). Affinity purification of polyclonal antibodies using a new all-D synthetic peptide ligand: comparison with protein A and protein G. J Immunol Methods 271, 77-88.
86. Verkhivker, G. M., Bouzida, D., Gehlhaar, D. K., Rejto, P. A., Freer, S. T., and Rose, P. W. (2002). Monte Carlo simulations of the peptide recognition at the consensus binding site of the constant fragment of human immunoglobulin G: the energy landscape analysis of a hot spot at the intermolecular interface. Proteins 48, 539-557.
87. Yang, H., Gurgel, P. V., and Carbonell, R. G. (2005). Hexamer peptide affinity resins that bind the Fc region of human immunoglobulin G. J Pept Res 66 Suppll, 120-137.
88. Yang, H., Gurgel, P. V., Williams, D. K., Jr., Bobay, B. G., Cavanagh, J., Muddiman, D. C., and Carbonell, R. G. (2010). Binding site on human immunoglobulin G for the affinity ligand HWRGWV. J Mol Recognit 23, 271-282.
89. Yoo, H. S., Lee, K. H., Oh, J. E., and Park, T. G. (2000). In vitro and in vivo anti-tumor activities of nanoparticles based on doxorubicin-PLGA conjugates. J Control Release 68, 419-431.
90. Zacharie, B., Fortin, D., Wilb, N., Bienvenu, J. F., As selin, M., Grouix, B., and Penney, C. (2009). 2,6,9-Trisubstituted purine derivatives as protein A mimetics for the treatment of autoimmune diseases. Bioorg Med Chem Lett 19, 242-246.
91. Ducry and Stump (2010) Antibody-Drug Conjugates: Linking Cytotoxic Payloads to Monoclonal Antibodies. Bioconjugate Chem. 21: 5-13.
92. Allen T M (2002) Ligand-Targeted Therapeutics in Anticancer Therapy. Nature Reviews 2: 750-763.
93. Brandtzaeg, P. (1983). Immunohistochemical characterization of intracellular J-chain and binding site for secretory component (SC) in human immunoglobulin (Ig)-producing cells. Mol Immunol 20, 941-966.
94. Makiya, R., and Stigbrand, T. (1992). Placental alkaline phosphatase has a binding site for the human immunoglobulin-G Fc portion. Eur J Biochem 205, 341-345.
95. Akilesh, S., Christianson, G. J., Roopenian, D. C., and Shaw, A. S. (2007). Neonatal FcR expression in bone marrow-derived cells functions to protect serum IgG from catabolism. J Immunol 179, 4580-4588.
96. Fridman, W. H., Neauport-Sautes, C., Daeron, M., Yodoi, J., Lowy, I., Brezin, C., Vaquero, C., Gelabert, M. J., and Theze, J. (1984). Induction of Fc receptors and immunoglobulin-binding factors in T-cell clones. Mol Immunol 21, 1243-1251.
97. Kabir, S. (2002). Immunoglobulin purification by affinity chromatography using protein A mimetic ligands prepared by combinatorial chemical synthesis. Immunol Invest 31, 263-278.
98. Nielsen, A. L., Jorgensen, F. S., Olsen, L., Christensen, S. F., Benie, A. J., Bjornholm, T., and St Hilaire, P. M. (2010). A diversity optimized combinatorial library for the identification of Fc-fragment binding ligands. Biopolymers 94, 192-205.
99. Yang, H., Gurgel, P. V., and Carbonell, R. G. (2009a). Purification of human immunoglobulin G via Fc-specific small peptide ligand affinity chromatography. J Chromatogr A 1216, 910-918.
100. Teng, S. F., Sproule, K., Husain, A., and Lowe, C. R. (2000). Affinity chromatography on immobilized “biomimetic” ligands. Synthesis, immobilization and chromatographic assessment of an immunoglobulin G-binding ligand. J Chromatogr B Biomed Sci Appl 740, 1-15.
101. Zacharie, B., Abbott, S. D., Bienvenu, J. F., Cameron, A. D., Cloutier, J., Duceppe, J. S., Ezzitouni, A., Fortin, D., Houde, K., Lauzon, C., et al. (2010). 2,4,6-trisubstituted triazines as protein a mimetics for the treatment of autoimmune diseases. J Med Chem 53, 1138-1145.

1. A method of identifying pre-neoplastic or neoplastic tissue of a mammal, the method comprising detecting autoantibodies complexed to antigen at the tissue, wherein an increase in the amount of autoantibodies complexed to antigen at the tissue, as compared to that at a control tissue, is indicative that the tissue is pre-neoplastic or neoplastic.

2. The method of claim 1, wherein the tissue is in the body of the mammal.

3. The method of claim 1, wherein the detecting is done intra-operatively.

4. The method of claim 1, wherein the detecting is done prior to surgery.

5. The method of claim 1, wherein the tissue is tissue that has been explanted from the mammal.

6. The method of claim 5, wherein the explant is selected from the group consisting of a fresh explant, a frozen explant, and a fixed explant.

7. The method of claim 1, wherein the autoantibodies are labeled with a label.

8. The method of claim 7, wherein the label on the antibody is selected from the group consisting of an optical reporter, a positron-emission tomography reporter, a magnetic resonance imaging reporter, and a biochemical marker.

9. The method of claim 1, wherein the autoantibodies are detected using an antibody detection reagent.

10. The method of claim 9, wherein the antibody detection reagent is selected from the group consisting of Anti-IgG, Protein A, Protein G, an anti-IgG, Fab(2) fragment of an anti-IgG, Fab(1) of an anti-IgG and a humanized mouse anti-human IgG, peptides that bind to the Fc region of antibodies, small molecules that recognize IgG for the species of interest, and small molecules that bind the Fc region.

11. The method of claim 1, wherein the autoantibodies and tissue are autologous.

12. The method of claim 1, wherein the autoantibodies and tissue are heterologous but are from one or more individuals of the same species.

13. The method of claim 1, wherein the mammal is a human.

14. The method of claim 1, wherein the pre-neoplastic or neoplastic tissue is in the liver.

15. The method of claim 14, wherein the neoplastic tissue is a fibrolamellar hepatocellular carcinoma.

16. The method of claim 1, wherein the pre-neoplastic or neoplastic tissue is tissue selected from the group consisting of liver, skin, breast, and prostate tissue.

17. The method of claim 16, wherein the neoplastic tissue is a liver, skin, breast or prostate cancer.
